(19) United States
(12) Patent Application Publication
(10) Pub. No.: US 2025/0288536 A1
(43) Pub. Date: Sep. 18, 2025

(54) PROTEIN PARTICLES INCLUDING AN ACTIVE AGENT AND METHODS OF MAKING AND USING THE SAME

(71) Applicants: Dairy Mangement Inc., Rosemont, IL (US); Corell University, Ithaca, NY (US)

(72) Inventors: Hongmin Dong, Ithaca, NY (US); Younas Dadmohammadi, Ithaca, NY (US); Alireza Abbaspourrad, Ithaca, NY (US); Lixin Yang, Ithaca, NY (US); Tiantian Lin, Ithaca, NY (US); Gopinathan H. Meletharayil, Palatine, IL (US); Emil S. Nashed, North Haledon, NJ (US); Rohit Kapoor, Elk Grove Village, IL (US)

(21) Appl. No.: 18/865,710

(22) PCT Filed: May 16, 2023
(86) PCT No.: PCT/US2023/067048

§ 371 Date: Nov. 14, 2024

Related U.S. Application Data
(60) Provisional application No. 63/342,288, filed on May 16, 2022.

(51) Int. Cl. A61K 9/51; A61K 8/02; A61K 8/49; A61K 8/64; A61K 31/405

(52) U.S. Cl. CPC A61K 9/5169; A61K 8/0241; A61K 8/492; A61K 8/64; A61K 31/405

(57) ABSTRACT

Described herein are particles including a protein and an active agent such as a particle comprising α-lactalbumin and tryptophan. Also described herein are methods of making and using particles that include a protein and an active agent.

### STATEMENT REGARDING ELECTRONIC FILING OF A SEQUENCE LISTING

A Sequence Listing in XML text format, entitled 1213-4WO_ST26.xml, 4,507 bytes in size, generated on Apr. 25, 2023, and filed herewith, is hereby incorporated by reference into the specification for its disclosures.

### FIELD

This invention relates to particles including a protein and an active agent and to methods of making and using such particles.

### BACKGROUND

Tryptophan (Trp) is a nonpolar aromatic essential amino acid that can be obtained exclusively from dietary intake. It contributes to normal growth, protein synthesis, and the synthesis of important neurotransmitters, and biological molecules. Tryptophan plays a key role in regulating neurobehavioral effects such as appetites, mood, sleep, and pain perception. Tryptophan and tryptophan-containing peptides have demonstrated various bioactive properties that are related to disease management, including psychological/cognitive function and being as antihypertensive, antioxidant, antidiabetic, and satiating agents. However, tryptophan usage is limited due to its stability in a food matrix and notable bitter taste because of its nonpolar and aromatic residues. Among the free amino acids, tryptophan has the lowest bitter taste threshold (BTT: 4 mmol/L) (Di Pizio & Nicoli, 2020 Molecules (Basel, Switzerland), 25(20), doi.org/10.3390/molecules25204623). It is noteworthy to mention that tryptophan is susceptible to be oxidated and degraded due to pH and temperature changes, thus affecting its bioactivities. Maillard reactions occur between the primary amino groups of tryptophan and reducing carbohydrates following heat treatment. In addition, tryptophan may be further degraded by oxidative species that are generated during Maillard reactions. Similarly, lipid-derived oxidative products have been reported to reduce the bioavailability of tryptophan in storage studies (Nielsen et al., 1985).

### SUMMARY OF THE INVENTION

A first aspect of the present invention is directed to a particle comprising: a protein; and an active agent, wherein the active agent is present within the protein (e.g., within the tertiary structure of the protein). In some embodiments, the active agent is nonspecifically bound (e.g., via hydrophobic interaction, electrostatic interaction, hydrogen bonding, etc.) to the protein.

A second aspect of the present invention is directed to a plurality of particles, wherein each particle of the plurality of particles comprises a protein and an active agent, wherein the active agent is present within the protein (e.g., within the tertiary structure of the protein). In some embodiments, the active agent is nonspecifically bound (e.g., via hydrophobic interaction, electrostatic interaction, hydrogen bonding, etc.) to the protein.

Another aspect of the present invention is directed to a composition comprising a carrier (e.g., water and/or an oil) and a particle of the present invention.

A further aspect of the present invention is directed to a method for preparing a particle, the method comprising: homogenizing and/or sonicating a composition comprising a protein and an active agent for about 1 minute to about 2 hours, wherein the composition has a pH of about 10 to about 12, thereby providing the particle.

A further aspect of the present invention is directed to a food product comprising a particle of the present invention.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 is a schematic of an exemplary method for forming an α-Lactalbumin-Tryptophan (α-La-Trp) complex according to some embodiments of the present invention.

FIG. 2 shows graphs of particle size distributions for α-La-Trp complexes at a α-La to Trp weight ratio of 20:1 by intensity, volume, and number, as measured at pH 11 and pH 7 each at 25° C.

FIG. 3 shows graphs of particle size distributions for α-La-Trp complexes at a α-La to Trp weight ratio of 15:1 by intensity, volume, and number, as measured at pH 11 and pH 7 each at 25° C.

FIG. 4 shows graphs of particle size distributions for α-La-Trp complexes at α-La to Trp weight ratio of 10:1 by intensity, volume, and number, as measured at pH 11 and pH 7 each at 25° C.

FIG. 5 shows graphs of particle size distributions for α-La-Trp complexes at a α-La to Trp weight ratio of 5:1 by intensity, volume, and number, as measured at pH 11 and pH 7 each at 25° C.

FIG. 6 is a graph of mean size (diameter of a particle in nm (d.nm)) (open square) and PDI (polydisperse index, solid square) of α-La-Trp particles prepared under various pressure conditions with an α-La and Trp weight ratio of 20:1 at pH 11 for 30 minutes.

FIG. 7 is a graph of mean size (d.nm) (open square) and PDI (polydisperse index, solid square) of α-La-Trp particles prepared with various recirculation times with α-La and Trp weight ratio of 20:1 under a pressure of 30000 psi at pH 11.

FIG. 8 shows graphs of particle size distributions for α-La-Trp complex nanoparticles formed at a weight ratio of 5:1 with: 30000 psi for 40 minutes (run 10), 20000 psi for 30 min (run 12), and 40000 psi for 30 min (run 14) by intensity and volume, as measured at pH 11 and pH 7 each at 25° C.

FIG. 9 shows graphs of particle size distributions for α-La-Trp complex nanoparticles formed at a weight ratio of 5:1 with 30000 psi for 40 minutes by intensity, volume and number, as measured at pH 11 and pH 7 each at 25° C.

FIG. 10 shows graphs from the particle stability analysis of α-La-Trp complex nanoparticles formed at a weight ratio of 5:1 with 30000 psi for 40 minutes. Panels A and B of FIG. 10 show the effect of storage time on particle size at pH 11 and pH 7, respectively; and Panels C and D of FIG. 10 show the effect of temperature on the particle size at pH 11 and pH 7, respectively.

FIG. 11 is another schematic of an exemplary method for forming an α-Lactalbumin-Tryptophan (α-La-Trp) complex according to some embodiments of the present invention.

FIG. 12 shows graphs of particle size distributions for α-lactalbumin (α-La) before (panels A1-A3 of FIG. 12) and after (panels B1-B3 of FIG. 12) ultrasonication treatment by intensity, volume and number, as measured at pH 11 and pH 7 each at 25° C.

FIG. 13 shows graphs of particle size distributions for tryptophan (Trp) before (panels A1-A3 of FIG. 13) and after (panels B1-B3 of FIG. 13) ultrasonication treatment by intensity, volume and number, as measured at pH 11 and pH 7 each at 25° C.

FIG. 14 shows graphs of particle size distributions α-La-Trp before (panels A1-A3 of FIG. 14) and after (panels B1-B3 of FIG. 14) ultrasonication treatment by intensity, volume and number, as measured at pH 11 and pH 7 each at 25° C.

FIG. 15 is an illustration of an exemplary homogenizer set up according to some embodiments of the present invention.

FIG. 16 shows graphs of ABTS scavenging activity of α-lactalbumin (panel A of FIG. 16), tryptophan (panel B of FIG. 16), and α-lactalbumin-tryptophan (panel C of FIG. 16) before and after HPH treatment.

FIG. 17 is a graph of the intrinsic fluorescence of a lactalbumin-tryptophan complex before and after high-pressure homogenization (HPH).

FIG. 18 shows graphs of the mean sizes (open square) and PDI (polydisperse index, solid square) of α-LA-Trp-NPs prepared at different conditions. Panel A of FIG. 18 is a graph of the effect of pressure (with α-LA/Trp ratio of 20:1 at pH 11, recirculation 30 min). Panel B of FIG. 18 is a graph of the effect of α-LA/Trp ratios (under HPH pressure of 206.8 MPa for 30 min). Panel C of FIG. 18 is a graph of the effect of recirculation time (with α-LA/Trp ratio of 5:1 at pH11, under HPH pressure of 206.8 MPa).

FIG. 19 shows graphs of the particle size distributions of α-LA, Trp, and α-LA-Trp mixtures without HPH at pH 11 and those with pH shifting from 11 to 7 and 11 to 3, 25° C. α-La (α-lactalbumin), Trp (tryptophan).

FIG. 20 shows graphs of fluorescence intensity as a result of HPH-induced self-assembly of α-LA and Trp (Panel A of FIG. 20) and fluorescence and turbidity changes during extended treatment with HPH (Panel B of FIG. 20).

FIG. 21 shows the JMP output for effect summary of all factors and factor combinations that were selected by the model, ranked based on their respective p-values (Panel A of FIG. 21) and prediction profiler and desirability plots showing the effects of independent variables on α-LA-Trp-NPs size (Y1) and Trp fluorescence intensity (Y2) at pH 11, and those at pH 7 (Y3 and Y4) (Panel B of FIG. 21).

FIG. 22 shows graphs of fluorescence spectroscopy (excitation wavelength of 295 nm) of the α-LA, Trp, and α-LA-Trp mixture with and without HPH treatment at pH 11 and pH 7 (Panel A of FIG. 22), a schematic of a proposed mechanism α-LA-Trp-NPs formation based on fluorescence data (Panel B of FIG. 22), and Scanning Electron Micrographs of α-LA (Panel C, images C1 and C4 of FIG. 20), Trp (Panel C, images C2 and C5 of FIG. 20), and α-LA-Trp (Panel C, images C3 and C6 of FIG. 20) mixture after HPH treatment.

FIG. 23 shows graphs of the particle size distributions of α-LA-NPs, Trp-NPs, and α-LA-Trp-NPs formed by HPH treatment at pH 11, 7, and 3, at 25° C.

FIG. 24 shows particle thermal stability analysis of α-LA-NPs, Trp-NPs, and α-LA-Trp-NPs after HPH treatment. NPs were formed at an α-LA/Trp ratio of 5:1, 206.8 MPa, 40 minutes at pH 11, and, in samples with pH shifting, the pH was shifted from 11 to 7.

FIG. 25 shows graphs of particle freeze-thaw and freeze-thaw-thermal stability analysis of α-LA-NPs, Trp-NPs, and α-LA-Trp-NPs made through HPH treatment. NPs were formed at α-LA/Trp ratio of 5:1, 206.8 MPa, 40 min at pH 11, and, in samples with pH shifting, the pH was shifted from 11 to 7 and 3.

FIG. 26 shows SEM images of freeze-dried α-LA (Panel A, image A1 of FIG. 26), Trp (Panel A, image A2 of FIG. 26), and the mixture of α-LA and Trp (Panel A, image A3 of FIG. 26) without high-pressure homogenization treatment (NOHPH) and with high-pressure homogenization treatment (HPH) (Panel A, images A4-A6 of FIG. 26), and graphs showing particle size distribution and PDI values (in parenthesis) of α-LA, Trp and the mixture of α-LA and Trp after freeze-drying and redispersed in water and PBS buffer, respectively (Panel B of FIG. 26).

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting to the invention.

All publications, patent applications, patents, and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B, and C, it is specifically intended that any of A, B, or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein is merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

A “portion” or “fragment” of a nucleotide sequence or polypeptide (including a domain) will be understood to mean a nucleotide sequence or polypeptide of reduced length (e.g., reduced by 1, 2, 3, 4, 5, 6, 7, 8, 9, 10, 11, 12, 13, 14, 15, 16, 17, 18, 19, 20 or more residue(s) (e.g., nucleotide(s) or peptide(s)) relative to a reference nucleotide sequence or polypeptide, respectively, and comprising, consisting essentially of and/or consisting of a nucleotide sequence or polypeptide of contiguous residues, respectively, identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleotide sequence or polypeptide.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Provided according to embodiments of the present invention are particles that comprise a protein and an active agent that is associated with the protein. In some embodiments, an active agent is present within a protein present in a particle of the present invention and/or an active agent is present on a surface of a protein present in a particle of the present invention. In some embodiments, the active agent is present within the tertiary structure of the protein. In some embodiments, the active agent is nonspecifically bound, such as via a hydrophobic interaction, electrostatic interaction, hydrogen bonding, and/or the like, to the protein. One or more (e.g., 1, 2, 3, 4, 5, 6, 7, 8, 9, 10, 15, 20, 25, 30, or more) protein molecule(s) (e.g., protein monomer(s)) may be present in a particle of the present invention. In some embodiments, one or more (e.g., 1, 2, 3, 4, 5, 6, 7, 8, 9, 10, 15, 20, 25, 30, or more) active agent(s) are present between two or more protein molecules that are associated with one another (e.g., via a non-specific interaction). In some embodiments, a particle of the present invention is a homogenous polygonal particle.

A particle of the present invention may comprise one or more (e.g., 1, 5, 10, 20, 30, 40, 50 or more) active agent(s), which may be the same or different from each other, and/or one or more (e.g., 1, 5, 10, 20, 30, 40, 50 or more) protein molecule(s), which may be the same or different from each other. In some embodiments, a particle of the present invention comprises an active agent that is within an area of the tertiary structure (e.g., within a tertiary fold) of the protein. In some embodiments, the active agent is present within an area of the tertiary structure (e.g., within a tertiary fold) of the protein that comprises at least one nonspecific hydrophobic interaction between two or more amino acid residues. In some embodiments, the active agent is present in a hydrophobic pocket of the protein. In some embodiments, the active agent is within the protein core of the protein. In some embodiments, the active agent is within a folded region of the protein that optionally has zero solvent accessibility. In some embodiments, when a particle of the present invention comprises a plurality of active agents (where the active agents in the plurality of active agents may be the same or different from each other), at least one active agent of the plurality of active agents is present within the protein and one or more of the active agent(s) of the plurality of active agents may be present on a surface of the protein.

Exemplary proteins of the present invention include, but are not limited to, dairy proteins (e.g., milk proteins), plant proteins, and/or animal (e.g., meat) proteins. A “dairy protein,” “milk protein,” “plant protein,” “animal protein,” and “meat protein,” as used herein refer to a protein that is found naturally in a dairy product, milk, plant, animal, and meat, respectively, and/or that is derived from such a naturally occurring protein to have an amino acid sequence having at least 70% sequence identity to the naturally occurring protein's amino acid sequence. For example, in some embodiments, a dairy, milk, plant, animal, or meat protein is naturally found in a dairy product, milk, plant, animal, or meat, respectively, and/or the protein is isolated from the dairy product, milk, plant, animal, or meat, respectively, or the protein is synthetically prepared to have an amino acid sequence having at least 70% sequence identity to the naturally occurring protein's amino acid sequence. In some embodiments, the protein is a milk protein such as, but not limited to, α-lactalbumin, β-lactoglobulin, and/or lactoferrin. In some embodiments, whey protein isolate (WPI), which comprises α-lactalbumin and β-lactoglobulin, is used to prepare a particle of the present invention, and a particle of the present invention comprises one or more of α-lactalbumin and one or more of β-lactoglobulin.

A protein that is used to prepare a particle of the present invention may have a molten globule state and/or have a bilobal structure. The protein may comprise two or more (e.g., 2, 3, 4, 5, or more) domains and/or the protein, at a pH of about 5 to about 9, may comprise one or more (e.g., 1, 2, 3, 4, or more) intramolecular disulfide bonds. In some embodiments, the protein comprises at least two domains and the protein comprises at least one disulfide bridge that connects two domains of the protein. In some embodiments, the protein is monomeric. In some embodiments, the protein has about 100 amino acids to about 200, 300, 400, or 500 amino acids and/or a molecular weight of about 10,000 kDa to about 20,000, 30,000, 40,000, or 50,000 kDa. The protein may have about 100, 150, 200, 250, 300, 350, 400, 450, or 500 amino acids. In some embodiments, the protein has a molecular weight of about 10,000, 15,000, 20,000, 25,000, 30,000, 35,000, 40,000, 45,000, or 50,000 kDa. The protein may have an isoelectric point (pI) of about 4, 4.1, 4.2, 4.3, or 4.4 to about 4.5, 4.6, 4.7, 4.8, 4.9, or 5. In some embodiments, the protein has a pI of about 4.2 to about 4.5. The protein may have a tertiary structure that comprises α-helices in an amount of about 10% or 15% to about 20%, 25%, or 30% of the total tertiary structure (optionally calculated by the percentage of the number of amino acids present in an α-helix to the total number of amino acids in the protein), β-sheets in an amount of about 1% or 5% to about 10%, 15%, 20%, or 25% of the total tertiary structure (optionally calculated by the percentage of the number of amino acids present in a β-sheet to the total number of amino acids in the protein), and/or an unordered tertiary structure in an amount about 50%, 55%, or 60% to about 65%, 70%, or 75% of the total tertiary structure (optionally calculated by the percentage of the number of amino acids present in an unordered, tertiary structure to the total number of amino acids in the protein).

A particle of the present invention may comprise a plurality of proteins. In some embodiments, a particle of the present invention comprises about 5, 10, or 20 to about 25, 30, 40, or 50 protein molecules (e.g., protein monomers). In some embodiments, a particle of the present invention comprises about 5, 10, 15, 20, 25, 30, 35, 40, 45, or 50 protein molecules (e.g., protein monomers). A particle of the present invention may comprise a protein in a total amount (e.g., one or more protein molecules) of about 75%, 80%, 85%, to about 90%, 95%, 99%, or 100% by weight of the particle and the active agent in a total amount (e.g., one or more active agents) of about 0.1%, 0.5%, 1% or 5% to about 10%, 15%, 20%, or 25% by weight of the particle.

Further exemplary proteins that may be present in a particle of the present invention include, but are not limited to, α-lactalbumin, lysozyme, cytochrome c, apomyoglobin, staphylococcal nuclease, α-lactoglobulin, lactoferrin, and any combination thereof. In some embodiments, a particle of the present invention comprises α-lactalbumin (e.g., bovine α-lactalbumin and/or human α-lactalbumin). A protein of the present invention may be from any source (e.g., plant, animal, etc.). In some embodiments, the protein is obtained and/or derived from an animal source such as a mammal (e.g., a bovine or human). In some embodiments, a particle of the present invention comprises about 5, 10, 15, 20, 25, 30, 35, 40, 45, or 50 α-lactalbumin molecules. In some embodiments, a protein present in a particle of the present invention has an amino acid sequence having about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to one or more of SEQ ID NOs: 1-3. In some embodiments, a protein present in a particle of the present invention has an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to one or more of SEQ ID NOs: 1-3. In some embodiments, a protein present in a particle of the present invention has an amino acid sequence having about 100% sequence identity to one or more of SEQ ID NOs: 1-3.

An active agent used to prepare a particle of the present invention may be an organic compound such as, but not limited to, an amino acid. In some embodiments, the active agent has a molecular weight of about 70, 100, 150, or 200 g/mol to about 250, 300, 400, or 500 g/mol. The active agent may have a solubility in water at 25° C. of about 15 mg/mL or less such as about 15, 14, 13, 12, 11, 10, 9, 8, 7, 6, or 5 mg/L, or less. In some embodiments, the active agent may have a solubility in water at 25° C. of about 5, 6, 7, 8, 9, or 10 mg/mL to about 11, 12, 13, 14, or 15 mg/L. In some embodiments, the active agent has a pKa of about 1.5, 2, 2.5, 2.6, 2.7, or 2.8 to about 2.9, 3, 3.2, or 3.5 and/or a pI of about 5, 5.5, 5.6, 5.7, or 5.8 to about 5.9, 6, 6.1, 6.2, 6.3, 6.4, or 6.5. Exemplary active agents include, but are not limited to, amino acids (e.g., tryptophan, leucine, phenylalanine, cysteine, and/or tyrosine), vitamin E, and any combination thereof. In some embodiments, the active agent present in a particle of the present invention is tryptophan.

A particle of the present invention may have a size (e.g., a diameter) in at least one dimension of about 25, 50, 75, 100, 125, or 150 nm to about 200, 250, 300, 350, 400, 450, 500, 550, 600, 650, 700, 750, 800, 850, or 900 nm, optionally as measured using microscopy (e.g., scanning electron microscopy (SEM) and/or transmission electron microscopy (TEM)) and/or dynamic light scattering (DLS). In some embodiments, the particle has a size (e.g., a diameter) in at least one dimension of about 25, 50, 75, 100, 125, 150, 200, 250, 300, 350, 400, 450, 500, 550, 600, 650, 700, 750, 800, 850, or 900 nm. In some embodiments, the particle has a size (e.g., a diameter) in at least one dimension of about 230 nm. In some embodiments, the particle is a nanoparticle. In some embodiments, a plurality of particles of the present invention, particles prepared according to a method of the present invention, and/or particles present in a composition of the present invention have a Dv (50) of about 175 or 200 nm to about 225, 250, 275, 300, or 325 nm, optionally as measured using microscopy (e.g., SEM and/or TEM) and/or DLS. In some embodiments, a plurality of particles of the present invention, particles prepared according to a method of the present invention, and/or particles present in a composition of the present invention have a Dv (50) of about 175, 200, 225, 250, 275, 300, or 325 nm, optionally as measured using microscopy (e.g., SEM and/or TEM) and/or DLS. In some embodiments, a plurality of particles of the present invention have a polydispersity index (PDI) of less than about 0.5 (e.g., of less than 0.5, 0.4, 0.3, 0.2, or 0.1). In some embodiments, a plurality of particles of the present invention have a PDI of less than 0.3 or 0.2.

In some embodiments, a protein and/or an active agent that are used to prepare a particle of the present invention are dissolved in water at a temperature of about 25° C. and a pH of about 11. In some embodiments, the active agent and/or protein dissolve in water at a temperature of about 25° C. and a pH of about 11 in an amount of about 25, 30, 40, or 45 mg/mL to about 50, 100, 150, 200, 250, 300, 350, or 400 mg/L. The protein and/or active agent may have a negative charge in water at a pH of about 11.

A particle of the present invention comprising a protein and an active agent may have improved (e.g., increased) storage, stability, activity, and/or function compared to the storage, stability, activity, and/or function of the protein alone (e.g., the protein not present in a particle of the present invention and not in association with the active agent). In some embodiments, upon storage at about 4° C. to about 10° C. in a closed container for about 1, 2, 3, 4, 5, or 6 month(s), the size (e.g., diameter) in at least one dimension of the particle remains within ±about 20% of its original size (e.g., the size at initial formation of the particle and/or the size at day 1 of storage). For example, at an initial time point (e.g., the start of day one of the storage time period), the particle may have a diameter of about 225 nm and after storage at about 4° C. to about 10° C. in a closed container for about 1, 2, 3, 4, 5, or 6 month(s) starting from day one of the storage time period, the particle may have a size that increased or decreased by about 20% or less. Thus, the particle having a starting size of about 225 nm may have a size at the end of the storage time period in a range of about 180 nm to about 270 nm. In some embodiments, upon storage at about 4° C. to about 10° C. in a closed container for about 1, 2, 3, 4, 5, or 6 month(s), a particle of the present invention has a size (e.g., diameter) in at least one dimension that is increased in an amount of less than about 20% compared to its original size. In some embodiments, a dried particle (e.g., a freeze-dried and/or spray-dried particle and/or a particle that comprises water in an amount of about 0% to about 5% by weight of the dried particle) is stored at about 4° C. to about 10° C. in a closed container for about 1, 2, 3, 4, 5, or 6 month(s) and optionally, at the end of the storage period, the size (e.g., diameter) of the dried particle is measured and/or the dried particle is re-constituted (e.g., dissolved and/or dispersed in) in a composition (e.g., water and/or a buffer) and the size (e.g., diameter) of the particle in the composition is measured. In some embodiments, a particle present in a composition (e.g., water and/or a buffer) is stored at about 4° C. to about 10° C. in a closed container for about 1, 2, 3, 4, 5, or 6 month(s) and optionally the size (e.g., diameter) of the particle in the composition is measured at the end of the storage time period.

In some embodiments, a particle of the present invention and/or a plurality of particles of the present invention is stable in that it has one peak particle size distribution in a composition at a pH of about 7 and/or in a composition at a pH of about 11, optionally as measured using microscopy (e.g., SEM and/or TEM) and/or DLS. In some embodiments, a particle of the present invention and/or a plurality of particles of the present invention is stable in that it has one peak particle size distribution in a composition at a pH of about 7 and one peak particle size distribution in a composition at a pH of about 11, optionally as measured using microscopy (e.g., SEM and/or TEM) and/or DLS. In some embodiments, a composition (e.g., a composition having a pH of about 3, 7, and/or about 11) comprising a plurality of particles has two or more peak particle size distributions, optionally as measured using microscopy (e.g., SEM and/or TEM) and/or DLS, which indicates that the particles of the plurality of particles are not stable. In some embodiments, a particle of the present invention is stable in that the particle does not fall out of (e.g., precipitate) and/or aggregate in a composition of the present invention. In some embodiments, a particle of the present invention is stable in that the particle does not fall out of (e.g., precipitate) and/or aggregate in a composition of the present invention when the pH of the composition is adjusted (e.g., from a pH of about 11 to a pH of about 7).

In some embodiments, a particle of the present invention has an increased freeze-thaw stability compared to the freeze-thaw stability of a protein alone (i.e., a protein not present in a particle of the present invention) and/or an active agent alone (i.e., an active agent not present in a particle of the present invention). “Freeze-thaw stability” as used herein can refer to the stability and/or properties of a material (e.g., a particle of the present invention, an active agent alone, or a protein alone) prior to and after exposure to a temperature at or below freezing (i.e., 0° C.) followed by exposure to a temperature above freezing (i.e., above 0° C.). Freeze-thaw stability may be determined by comparing the stability and/or properties of the material prior to freezing (i.e., prior to exposure to 0° C. or less) and the stability and/or properties of the same material after freezing and thawing (i.e., after exposure to 0° C. or less and exposure to above 0° C.). In some embodiments, one or more properties of a particle of the present invention prior to freezing and after exposure to a temperature above freezing may be compared to the same one or more properties of a protein alone prior to and after the same conditions, optionally wherein the protein is the same protein as present in the particle. In some embodiments, one or more properties of a particle of the present invention prior to freezing and after exposure to a temperature above freezing may be compared to the same one or more properties of an active agent alone prior to and after the same conditions, optionally wherein the active agent is the same active agent as present in the particle. In some embodiments, one or more properties of a. first particle of the present invention prior to freezing and after exposure to a temperature above freezing may be compared to the same one or more properties of a second particle of the present invention prior to and after the same conditions, optionally wherein the first and second particles comprise the same protein and/or active agent, but are different in some manner (e.g., prepared differently and/or have a different size and/or concentration of the protein and/or active agent). In some embodiments, freeze-thaw stability may be determined by comparing the stability and/or properties of the material prior to freezing (i.e., prior to exposure to 0° C. or less) and the stability and/or properties of the material after freezing and thawing (i.e., after exposure to 0° C. or less and exposure to above 0° C.). In some embodiments, freeze-thaw stability is determined by measuring the amount of change, if any, in particle size (e.g., diameter), polydispersity index, and/or particle size distribution, and/or by one or characteristics of the material as a dry product and/or in an aqueous composition (e.g., presence and/or the amount of precipitation and/or aggregates (e.g., aggregations) of the material).

In some embodiments, a particle of the present invention has an improved freeze-thaw stability. In some embodiments, after a change in temperature (e.g., a change in storage temperature, an increase in temperature, and/or a change in temperature from about −20, −15, or −10° C. to about 15, 20, or 25° C.), the size (e.g., diameter) in at least one dimension of a particle of the present invention, optionally at a pH of about 7 to about 11 (e.g., a pH of about 7 or about 11), remains within ±about 20% of its original size (e.g., the size at initial formation of the particle and/or the size prior to the change in temperature), optionally within about ±about 15% of its original size. For example, prior to a change in temperature from about −20, −15, or −10° C. to about 15, 20, or 25° C., the particle (optionally at a pH of about 7 or 11) may have a diameter of about 230 nm and, after the change in temperature from about −20, −15, or −10° C. to about 15, 20, or 25° C., the particle (optionally at a pH of about 7 or 11) may have a size that increased or decreased by about 20% or less, optionally a size that increased or decreased by about 15% or less. Thus, optionally at a pH of about 7 or 11, the particle having a size of 230 nm, prior to the change in temperature, may have a size after the change in temperature in a range of about 185 nm to about 275 nm. In some embodiments, after a change in temperature from about −20, −15, or −10° C. to about 15, 20, or 25° C., a particle of the present invention, optionally at a pH of about 7 or 11, has a size (e.g., diameter) in at least one dimension that is increased in an amount of less than about 20% compared to its original size, optionally increased in an amount of less than about 15% compared to its original size. In some embodiments, a particle of the present invention is and/or remains stable (e.g., the particle does not fall out of a composition (e.g., precipitate) and/or does not aggregate with other particles) prior to, during, and/or after a change in temperature (e.g., a change in temperature from about −20, −15, or −10° C. to about 15, 20, or 25° C.).

In some embodiments, a particle of the present invention has an increased freeze-thaw-thermal stability compared to the freeze-thaw-thermal stability of a protein alone (i.e., a protein not present in a particle of the present invention) and/or an active agent alone (i.e., an active agent not present in a particle of the present invention). “Freeze-thaw-thermal stability” as used herein can refer to the stability and/or properties of a material (e.g., a particle of the present invention, an active agent alone, or a protein alone) prior to and after exposure to a temperature at or below freezing (i.e., 0° C.) followed by exposure to a temperature above freezing (i.e., above 0° C.) that includes a heat treatment (i.e., an exposure to a temperature of at least 55° C. for at least 25 minutes) Freeze-thaw-thermal stability may be determined by comparing the stability and/or properties of the material prior to freezing (i.e., prior to exposure to 0° C. or less) and the stability and/or properties of the same material after freezing and thawing that includes a heat treatment (i.e., after exposure to 0° C. or less and exposure to above 0° C. that includes a heat treatment). In some embodiments, one or more properties of a particle of the present invention prior to freezing and after the heat treatment may be compared to the same one or more properties of a protein alone prior to and after the same conditions, optionally wherein the protein is the same protein as present in the particle. In some embodiments, one or more properties of a particle of the present invention prior to freezing and after the heat treatment may be compared to the same one or more properties of an active agent alone prior to and after the same conditions, optionally wherein the active agent is the same active agent as present in the particle. In some embodiments, one or more properties of a first particle of the present invention prior to freezing and after the heat treatment may be compared to the same one or more properties of a second particle of the present invention prior to and after the same conditions, optionally wherein the first and second particles comprise the same protein and/or active agent, but are different in some manner (e.g., prepared differently and/or have a different size and/or concentration of the protein and/or active agent). In some embodiments, freeze-thaw-thermal stability may be determined by comparing the stability and/or properties of the material prior to freezing (i.e., prior to exposure to 0° C. or less) and the stability and/or properties of the material after freezing and thawing that includes a heat treatment. In some embodiments, freeze-thaw-thermal stability is determined by measuring the amount of change, if any, in particle size (e.g., diameter), polydispersity index, and/or particle size distribution, and/or by one or characteristics of the material as a dry product and/or in an aqueous composition (e.g., presence and/or the amount of precipitation and/or aggregates (e.g., aggregations) of the material).

In some embodiments, a particle of the present invention has an improved freeze-thaw-thermal stability. In some embodiments, after a change in temperature (e.g., a change in storage temperature, an increase in temperature, and/or a change in temperature from about −20, −15, or −10° C. to about 15, 20, or 25° C.) followed by heat treatment (e.g., a heat treatment at about 55, 60, or 65° C. for about 25, 30, or 35 minutes), the size (e.g., diameter) in at least one dimension of the particle, optionally at a pH of about 7 to about 11 (e.g., at a pH of about 7 or 11), remains within ±about 20% of its original size (e.g., the size at initial formation of the particle and/or the size prior to the change in temperature), optionally within ±about 15% of its original size. For example, prior to the change in temperature that is followed by heat treatment, the particle may have a diameter of about 230 nm and after the change in temperature and heat treatment, the particle, optionally at a pH of about 7 or 11, may have a size that increased or decreased by about 20% or less, optionally a size that increased or decreased by about 15% or less. Thus, the particle, optionally at a pH of about 7 or 11, having a size of 230 nm, prior to the change in temperature and heat treatment, may have a size after the change in temperature and heat treatment in a range of about 170 nm to about 290 nm. In some embodiments, after a change in temperature from about −20, −15, or −10° C. to about 15, 20, or 25° C. followed by heat treatment at about 55, 60, or 65° C. for about 25, 30, or 35 minutes, a particle of the present invention, optionally at a pH of about 7 or 11, has a size (e.g., diameter) in at least one dimension that is increased in an amount of less than about 20% compared to its original size, optionally increased in an amount of less than about 15% compared to its original size. In some embodiments, a particle of the present invention is stable (e.g., the particle does not fall out of a composition (e.g., precipitate) and/or does not aggregate with other particles) when the particle is exposed to a change in temperature (e.g., a change in temperature from about −20, −15, or −10° C. to about 15, 20, or 25° C.) followed by a heat treatment (e.g., a heat treatment at about 55, 60, or 65° C. for about 25, 30, or 35 minutes). In some embodiments, a particle of the present invention has a synergistic effect. “Synergistic”, “synergy”, or grammatical variants thereof as used herein refer to a combination exhibiting an effect greater than the effect that would be expected from the sum of the effects of the individual parts of the combination alone. For example, the terms “synergistic” or “synergy” with regard to a particle of the present invention may refer to a combination of a protein and an active agent and/or may refer to two different method steps for preparing a particle of the present invention that result in a property and/or effect that is greater than that which would be expected from the sum of the individual parts alone. In some embodiments, a particle of the present invention prepared using a high-pressure homogenization step and a pH-shifting step may have a stability (e.g., a colloidal stability, pH stability, thermal stability, freeze-thaw stability, and/or freeze-thaw-thermal stability) that is greater than the sum of the stability of a particle including the same protein and active agent in the same amounts prepared with the same high-pressure homogenization alone and the stability of a particle including the same protein and active agent in the same amounts prepared with the same pH-shifting step alone.

In some embodiments, a particle of the present invention has an increased activity and/or function (e.g., increased antioxidant activity) compared to the activity and/or function of the protein alone. In some embodiments, a particle of the present invention has an improved function. In some embodiments, a particle of the present invention improves a property of the protein and/or the active agent present in the particle. For example, in some embodiments, a particle of the present invention may reduce the bitterness of an active agent (e.g., tryptophan) such as by increasing the bitter taste threshold (BTT), optionally as measured by Di Pizio & Nicoli, 2020 Molecules (Basel, Switzerland), 25 (20), doi.org/10.3390/molecules25204623, which is incorporated herein for methods of measuring bitterness and/or BTT. For example, a particle of the present invention may provide a BTT of greater than about 4 mmol/L for an active agent.

According to some embodiments, a composition comprising a particle of the present invention is provided. In some embodiments, the composition comprises a plurality of particles of the present invention. In some embodiments, the composition comprises a particle of the present invention and a carrier. The carrier may be a liquid such as, but not limited to, water and/or an oil. In some embodiments, the carrier is a food-grade component such as, but not limited to, an alcohol (e.g., ethanol such as in an amount of about 5% to about 20%). One or more excipient(s) may be present in a composition of the present invention. Exemplary excipients include, but are not limited to, pectins and/or gums. In some embodiments, a composition of the present invention is devoid of a masking agent, flavoring agent, cyclodextrin (e.g., β-cyclodextrin), and/or a physical barrier that is optionally configured to mask or reduce the taste of the active agent present in a particle of the present invention. In some embodiments, the composition is not a gel (e.g., a hydrogel such as a protein hydrogel) and/or is not an emulsion. In some embodiments, a particle of the present invention is not present in a gel (e.g., a hydrogel such as a protein hydrogel) or an emulsion. In some embodiments, a composition of the present invention is devoid of an agent (e.g., masking agent) that is configured and/or designed to reduce bitterness and/or off-taste of an active agent present in the composition rather than configured and/or designed for providing the desired flavor or taste of the composition.

In some embodiments, a composition of the present invention is a food product, nutritional supplement, therapeutic drink, and/or cosmetic. In some embodiments, a particle of the present invention may be present in a food product. In some embodiments, the food product is a dairy product (e.g., milk, yogurt, etc.).

An active agent present in a particle of the present invention may remain associated with (e.g., complexed with, within, etc.) the particle and/or protein present in the particle when present in a carrier and/or composition of the present invention. In some embodiments, about 30% or less of the total amount of active agent added to a carrier and/or composition is present free (i.e., not associated with the particle and/or protein) in the carrier and/or composition. For example, about 0%, 1%, 2%, 5%, 10%, or 15% to about 20%, 25%, or 30% of the active agent present in a particle of the present invention that is provided in a carrier and/or composition and/or that is used to prepare a particle of the present invention may be present in the carrier and/or composition as free active agent (i.e., active agent that is not associated with the particle and/or protein). Thus, for a particle comprising the active agent in a given amount, when the particle is added to a carrier and/or composition, the amount of free active agent present in the carrier and/or composition may be about 30% or less than the given amount of the active agent present in the particle. In some embodiments, a composition comprising water and particles of the present invention, which particles are present in an amount of about 100 mg of particles per mL of water, comprises free active agent in an amount of about 0%, 1%, 2%, 5%, 10%, or 15% to about 20%, 25%, or 30% by weight of the total amount of the active agent present in the particles. A composition of the present invention may be a dispersion (e.g., a colloidal dispersion). In some embodiments, a composition of the present invention has no visible aggregation in the composition (e.g., no visible clumps, aggregates, or particulates). In some embodiments, the composition is clear and is not cloudy or opaque. In some embodiments, the composition may appear turbid, but no sediments and/or aggregations are present.

Provided according to some embodiments of the present invention is a method for preparing a particle of the present invention. In some embodiments, the method comprises homogenizing and/or sonicating a composition comprising a protein and an active agent for about 1, 5, 10, 15, 20, or 30 minute(s) to about 40, 45, 50, 55, 60, 70, 80, 90, 100, 110, or 120 minutes, wherein the composition has a pH of about 10 or 10.5 to about 11, 11.5, or 12, thereby providing the particle. In some embodiments, the homogenizing and/or sonicating is carried out for about 10 minutes to about 50 minutes. The composition comprising the protein and the active agent may be homogenized and/or sonicated for about 1, 5, 10, 15, 20, 30, 40, 50, 60, 70, 80, 90, 100, 110, or 120 minute(s). In some embodiments, the composition has a pH of about 10, 10.5, 11, 11.5, or 12. In some embodiments, the composition has a pH of about 11. In some embodiments, the protein and/or active agent in the composition have a negative charge, optionally prior to, during, and/or after homogenizing and/or sonicating the composition. In some embodiments, the homogenizing and/or sonicating is carried out at a temperature in a range from about 15° C. or 20° C. to about 25° C. or 30° C., optionally at a temperature of about 15° C., 20° C., 25° C., or 30° C.

In some embodiments, a composition having a pH of about 10 or 10.5 to about 11, 11.5, or 12 (e.g., optionally about 11) that is used to prepare a particle of the present invention may comprise a protein and an active agent in a weight ratio of about 2:1, 3:1, 4:1, 5:1, 6:1, 7:1, 8:1, 9:1, or 10:1 to about 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1 (protein:active agent). In some embodiments, the composition comprises the protein and active agent in a weight ratio of about 5:1 to about 20:1 (protein:active agent). In some embodiments, the composition having a pH of about 10 or 10.5 to about 11, 11.5, or 12 (e.g., optionally about 11) that is used to prepare a particle of the present invention comprises a protein and an active agent in a weight ratio of about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1 (protein:active agent). In some embodiments, the composition having a pH of about 10 or 10.5 to about 11, 11.5, or 12 (e.g., optionally about 11) that is used to prepare the particle has a solids content of about 1%, 2%, 3%, 4%, 5%, 6%, 7%, 8%, 9%, or 10% w/v to about 11%, 12%, 13%, 14%, 15%, 16%, 17%, 18%, 19%, or 20% w/v. In some embodiments, the composition having a pH of about 10 or 10.5 to about 11, 11.5, or 12 (e.g., optionally about 11) that is used to prepare the particle has a solids content of about 1%, 2%, 3%, 4%, 5%, 6%, 7%, 8%, 9%, 10%, 11%, 12%, 13%, 14%, 15%, 16%, 17%, 18%, 19%, or 20% w/v.

In a composition having a pH of about 10 or 10.5 to about 11, 11.5, or 12 (e.g., optionally about 11) that is used to prepare a particle of the present invention, the active agent may be present in an amount of about 1, 5, 10, 15, 20, 25, 30, 35, 40, 45, or 50 mg/mL to about 55, 60, 65, 70, 75, 80, 85, 90, 95, 100, 150, or 200 mg/mL and/or the protein may be present in an amount of about 1, 5, 10, 15, 20, 25, 30, 35, 40, 45, 50, 55, 60, 65, 70, 75, 80, 85, 90, 95, or 100 mg/mL to about 110, 120, 130, 140, 150, 160, 170, 180, 190, 200, 210, 220, 230, 240, 250, 260, 270, 280, 290, or 300 mg/mL. For example, in some embodiments, the active agent (e.g., tryptophan) may be dissolved in a composition having a pH of about 11 in an amount of about 30 mg/mL to about 50 mg/mL and the protein (e.g., α-lactalbumin and/or WPI) may be dissolved in the same composition in an amount of about 50 or 100 mg/mL to about 200 or 300 mg/L. In some embodiments, α-lactalbumin is dissolved in a composition having a pH of about 11 in an amount of about 200 mg/mL or less such as in an amount of about 100 mg/mL to about 200 mg/mL. In some embodiments, WPI is dissolved in a composition having a pH of about 11 in an amount of about 50 mg/mL or less such as in an amount of about 10 mg/mL to about 50 mg/mL. In some embodiments, lactoferrin is dissolved in a composition having a pH of about 11 in an amount of about 10 mg/mL or less such as in an amount of about 1 mg/mL to about 10 mg/mL.

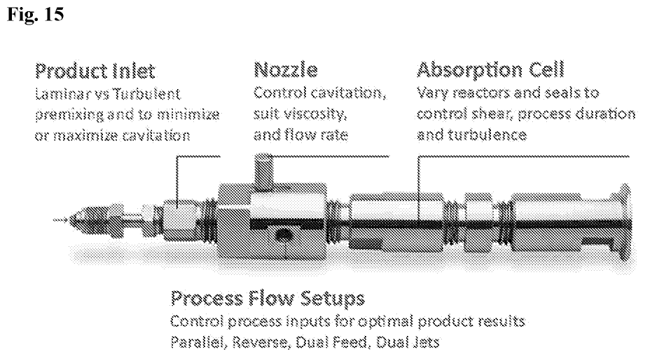

Homogenizing a composition having a pH of about 10 or 10.5 to about 11, 11.5, or 12 and comprising a protein and an active agent may be carried out with methods and/or homogenizers known in the art. In some embodiments, homogenizing a composition of the present invention comprises homogenizing the composition at a pressure of about 5,000, 10,000, 15,000, or 20,000 psi to about 25,000, 30,000, 35,000, 40,000, or 45,000 psi. In some embodiments, homogenizing the composition comprises homogenizing the composition at a pressure in a range of about 20,000 psi to about 40,000 psi. In some embodiments, homogenizing the composition comprises homogenizing the composition at a pressure of about 5,000, 10,000, 15,000, 20,000, 25,000, 30,000, 35,000, 40,000, or 45,000 psi. In some embodiments, a homogenizer used in a method of the present invention may comprise a device as shown in FIG. 15. In some embodiments, a homogenizer may be and/or comprise a device sold commercially by Bee International, USA, such as, but not limited to a BEE Emulsifying Cell sold by Bee International, USA. In some embodiments, a method of the present invention comprises homogenizing using a homogenizer and/or device that provides in-line cavitation. In some embodiments, a method of the present invention comprises homogenizing with a homogenizer and/or device in which a composition comprising a protein and an active agent is transmitted through a nozzle at a pressure (e.g., a pressure of about 5,000 psi to about 45,000 psi) to thereby provide a high-velocity jet stream (e.g., a jet stream having a velocity of up to about 70 mL/min). In some embodiments, transmitting and/or flowing the composition through an inlet of a homogenizer and/or device thereof to a nozzle, may be laminar, such as for a gentle process, or turbulent such as for pre-mixing. A nozzle of a homogenizer and/or device thereof may comprise an orifice having a diameter of about 0.1 mm, which may induce high shear. Transmission of a composition of the present invention through the nozzle may provide for sudden acceleration together with a pressure drop upon exiting the orifice of the nozzle, which can induce cavitation. After the composition exits the nozzle, the composition may flow and/or be transmitted into an absorption cell containing one or more orifices having a diameter of about 0.5 mm or more. Without being bound by any particular theory, the kinetic energy of the fluid jet in such a homogenization step may be absorbed into the composition and/or a particle(s) present in the composition optionally by manipulating the flow and/or translating its velocity into forces of shear, cavitation, and/or impact. In some embodiments, homogenizing a composition of the present invention comprises exposing the composition to high-pressure homogenization (HPH) and/or to high shear force, cavitation, and turbulence.

Sonicating a composition having a pH of about 10 or 10.5 to about 11, 11.5, or 12 and comprising a protein and an active agent may be carried out with methods and/or sonicators known in the art. In some embodiments, sonicating the composition comprises sonicating the composition at frequency of about 15 or 20 kHz to about 25 or 30 kHz with an amplitude of about 40%, 45%, or 50% to about 55%, 60%, 65%, or 70%. In some embodiments, sonicating the composition comprises sonicating the composition at frequency of about 15, 20, 25, or 30 kHz with an amplitude of about 40%, 45%, 50%, 55%, 60%, 65%, or 70%.

A method of the present invention may comprise, after homogenizing and/or sonicating the composition, adjusting the pH of the composition to a pH of about 6.5 to about 7.5. In some embodiments, the pH of the composition is adjusted to a pH of about 7. An acid and/or base (e.g., an organic acid and/or organic base) may be used to adjust the pH of the composition.

A method of the present invention may provide a particle of the present invention having a particle size distribution from about 50, 100, 200, or 300 nm to about 400, 500, 600, or 700 nm. In some embodiments, the particle may have a polydispersity index of less than about 0.5, 0.45, 0.4, 0.35, 0.3, 0.25, or 0.2. In some embodiments, the particle may have a mean particle size (e.g., a mean particle diameter) of about 100, 125, 150, or 175 nm to about 200, 225, 250, 275, or 300 nm. In some embodiments, the particle may have a mean particle size (e.g., a mean particle diameter) of about 100, 125, 150, 175, 200, 225, 250, 275, or 300 nm. In some embodiments, the particle may have a mean particle size (e.g., a mean particle diameter) of about 230 nm.

In some embodiments, a method of the present invention comprises dehydrating a particle of the present invention and/or a composition in which the particle is present. Dehydrating a particle of the present invention and/or a composition comprising a particle of the present invention may be carried out using methods and/or devices known in the art. In some embodiments, dehydrating a particle of the present invention and/or a composition comprising a particle of the present invention comprises freeze-drying and/or spray-drying the particle and/or composition.

A method of the present invention may comprise reducing the size (e.g., diameter) of a particle of the present invention. For example, in some embodiments, the size (e.g., diameter) of a particle of the present invention may be reduced upon adjusting the pH of a composition in which the particle is present. In some embodiments, a method of the present invention comprises reducing the size (e.g., diameter, optionally the average diameter) of a particle of the present invention by about 5%, 10%, 15%, or 20% to about 25%, 30%, 35%, or 40% compared to the size of the particle in a composition at a pH of about 11. In some embodiments, the size (e.g., diameter, optionally the average diameter) of a particle of the present invention present in a composition may be reduced by about 5%, 10%, 15%, or 20% to about 25%, 30%, 35%, or 40% after the pH of the composition is adjusted from a pH of about 11 to a pH of about 7.

In some embodiments, a method of the present invention comprises administering a therapeutically effective amount of a particle of the present invention and/or a composition of the present invention to a subject. As used herein, the term “therapeutically effective amount” refers to an amount of particle and/or composition of the present invention that elicits a therapeutically useful response in a subject. Those skilled in the art will appreciate that the therapeutic effects need not be complete or curative, as long as some benefit is provided to the subject.

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom associated with the subject's condition is achieved and/or there is a delay in the progression of the symptom. In some embodiments, the severity of a symptom associated with sleep quality and/or mental health may be reduced in a subject compared to the severity of the symptom in the absence of a method of the present invention. In some embodiments, a particle of the present invention and/or a composition of the present invention is administered to a subject to improve sleep quality (e.g., increase the length of sleep time and/or time in rapid eye movement (REM) sleep, reduces sleep interruptions, etc.), improve mental health, and/or treat a disease and/or a symptom thereof.

In some embodiments, a particle of the present invention and/or a composition of the present invention may be administered in a treatment effective amount. A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount may be achieved by administering a composition of the present invention.

The terms “prevent,” “preventing” and “prevention” (and grammatical variations thereof) refer to avoidance, reduction and/or delay of the onset of a symptom associated with a disease, disorder, or condition and/or a reduction in the severity of the onset of symptom associated with a disease, disorder, or condition relative to what would occur in the absence of a method of the present invention. The prevention can be complete, e.g., the total absence of the symptom. The prevention can also be partial, such that the occurrence of the symptom in the subject and/or the severity of onset is less than what would occur in the absence of a method of the present invention. In some embodiments, a particle of the present invention and/or a composition of the present invention is administered to a subject to prevent a disease, disorder, or condition.

In some embodiments, a particle of the present invention and/or a composition of the present invention may be administered in a prevention effective amount. A “prevention effective” amount as used herein is an amount that is sufficient to prevent (as defined herein) a symptom associated with a disease, disorder, or condition in a subject. Those skilled in the art will appreciate that the level of prevention need not be complete, as long as some benefit is provided to the subject. In some embodiments, a prevention effective amount may be achieved by administering a composition of the present invention.

The present invention finds use in both veterinary and medical applications. Subjects suitable to be treated with a method of the present invention include, but are not limited to, mammalian subjects. Mammals of the present invention include, but are not limited to, canines, felines, bovines, caprines, equines, ovines, porcines, rodents (e.g. rats and mice), lagomorphs, primates (e.g., simians and humans), non-human primates (e.g., monkeys, baboons, chimpanzees, gorillas), and the like, and mammals in utero. Any mammalian subject in need of being treated according to the present invention is suitable. Human subjects of both genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects. In particular embodiments of the present invention, the subject is a human adolescent and/or adult.

A method of the present invention may also be carried out on animal subjects, particularly mammalian subjects such as mice, rats, dogs, cats, livestock and horses for veterinary purposes, and/or for drug screening and drug development purposes.

In some embodiments, the subject is “in need of” or “in need thereof” a method of the present invention, for example, the subject has findings typically associated with a disease, disorder, or condition, is suspected to have a disease, disorder, or condition, and/or the subject has a disease, disorder, or condition.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

Bovine milk α-lactalbumin powder was kindly provided by Agropur, USA (batch number, JE 0001-21-414, 92.5% purity). Pure tryptophan was purchased from Sigma (St. Louis, MO, USA). All water used was Milli-Q water. All other chemicals were of analytical grade.

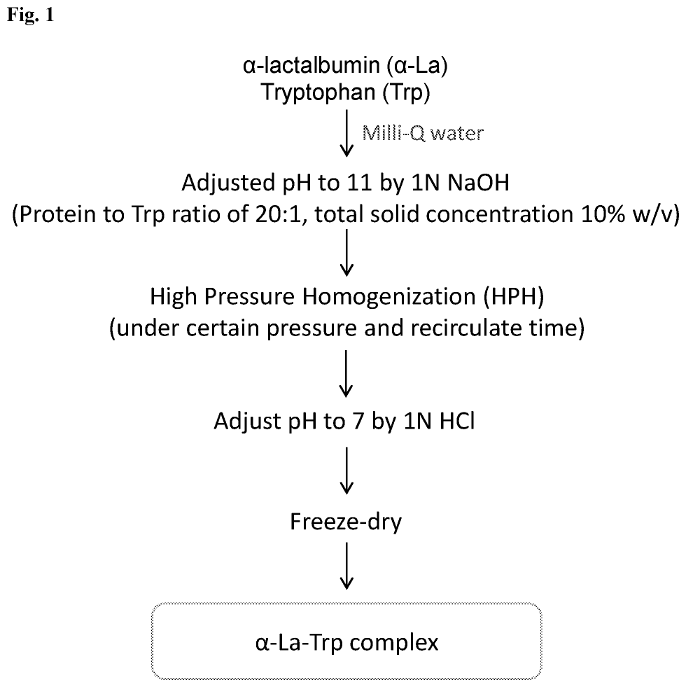

The procedure used in this example for the α-Lactalbumin-Tryptophan (α-La-Trp) complex formation is shown in FIG. 1. Trp was mixed with α-La at various α-La to Trp weight ratios (20:1, 15:1, 10:1, and 5:1). The mixture of α-La and Trp was fully dissolved in Milli-Q water by adjusting pH to 11 using 1N NaOH solution (final total solid concentration of α-La and Trp was 10%, w/v). The mixture was then passed through a high-pressure homogenizer (HPH) (Nano DeBEE, Bee International, USA) at pressures ranging from 1000 psi to 40000 psi, and homogenized (e.g., recirculated through the homogenizer) for 5-60 min. The collected α-La-Trp suspension was adjusted back to neutral pH (pH 7) using 1N HCl solution. The effect of the α-La to Trp ratios, HPH pressure, and circulation time on the complex formation was investigated based on the particle size, particle size distribution, and polydispersity index (PDI).

A Box-Behnken design (BBD) of 15 runs was applied to evaluate the formation conditions. Three extraction factors (α-La to Trp ratios, HPH pressure, and circulation time) were studied. Three equally spaced levels were selected for α-La to Trp weight ratios (i.e., 15:1, 10:1, and 5:1), pressure (i.e., 20000, 30000, and 40000 psi), and circulation time (i.e., 20, 30, and 40 min) as shown in Table 1. Mean particle size and PDI values at pH 11 and 7 were selected as responses of this study. The evaluation was done using JMP pro 16 (SAS Institute Inc. NC, USA).

TABLE 1
Variables and their levels in Box-Behnken design.

| | Levels | | |
|---|---|---|---|
| | −1 | 0 | +1 |
| Independent variables | (Low) | (Medium) | (High) |
| X1 = α-La to Trp ratio | 5:1 | 10:1 | 15:1 |
| X2 = Pressure (psi) | 20000 | 30000 | 40000 |
| X3 = Recirculate time | 20 | 30 | 40 |
| (min) | | | |
| Dependent variables | Goals | | |
| Y1 = Mean size (d · nm) | Minimize | | |
| at pH 11 | | | |
| Y2 = PDI at pH 11 | Minimize | | |
| Y3 = Mean size (d · nm) | Minimize | | |
| at pH 7 | | | |

| | |
|---|---|
| Y4 = PDI at pH 7 | Minimize |

Selected samples were used for stability analysis. Storage stability studies were carried out at 4° C. for 1, 7, 14, 21, and 28 days. Particle size and particle size distribution were measured. Color and physical state were illustrated visually by taking photos. Thermal stability of the complex was measured under different processing conditions: 63° C. for 30 min and 90° C. for 2 min based on the particle size, particle size distribution, and polydispersity index (PDI).

### 1. The Effect of α-La to Trp Weight Ratios on the Particle Size of α-La-Trp Complex

The effect of α-La to Trp weight ratios (20:1, 15:1, 10:1, and 5:1) on the particle size of α-La-Trp complex was performed under an HPH pressure of 30000 psi for 30 min. Clear differences were observed in the appearance of α-La and Trp mixture with and without HPH treatment. Without HPH treatment, the mixture appeared to be clear, whereas the mixture after HPH treatment had a cloudy/colloidal appearance indicating particle formation.

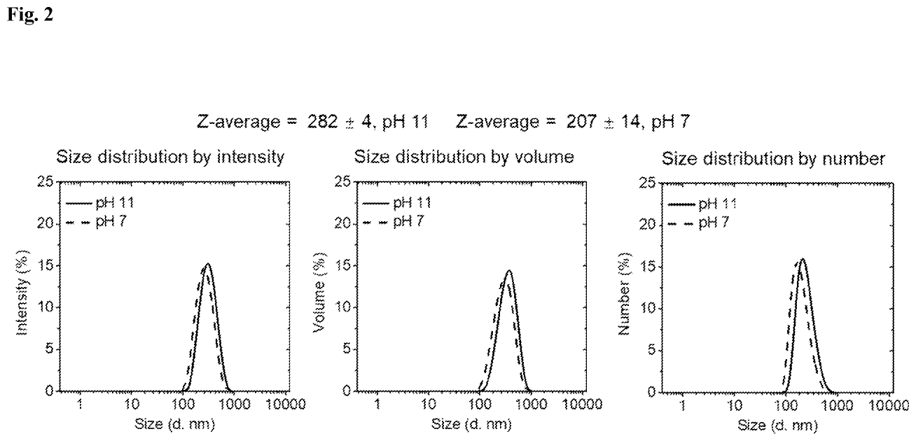

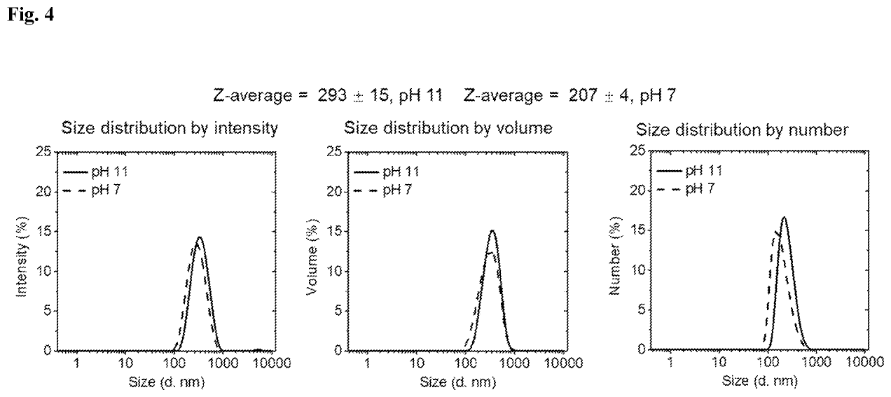

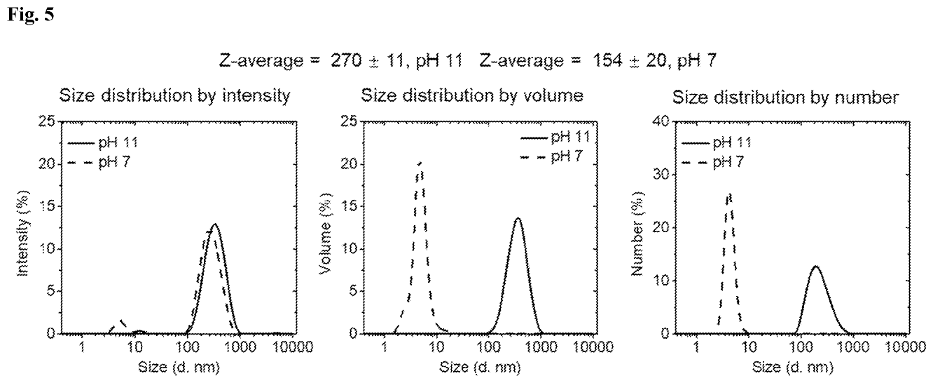

α-La-Trp complex nanoparticles exhibited a colloidal suspension without any visible aggregation. When measuring the particle size of the colloidal suspension by DLS (FIGS. 2-5), those samples produced by HPH at pH 11 show a narrow particle size distribution with mean particle size range from 270-293 nm for all the ratios tested. When adjusting the pH to 7, the particle size distribution for the ratios of 20:1, 15:1, and 10:1 shifted slightly to the smaller size range with mean particle size of ˜200 nm. While not wishing to be bound to any particular theory, this may be due to the molecular association (e.g., hydrophobic interaction, electrostatic interaction, hydrogen bonding) enhanced during the pH shifting. However, the particle size distribution for the ratio of 5:1 separated into two peaks, which, without wishing to be bound to any particular theory, may be due to the higher amount of tryptophan present during the formation of the particles compared to the other weight ratios. This indicated the particles formed at a ratio of 5:1 were stable at pH 11, while not stable at pH 7 using these particle formation conditions. Therefore, the lowest α-La to Trp weight ratio for the HPH condition of 30000 psi for 30 minutes should be 5:1 (α-La:Trp).

### 2. The Effect of Pressure and Recirculation Time of HPH on the Particle Size

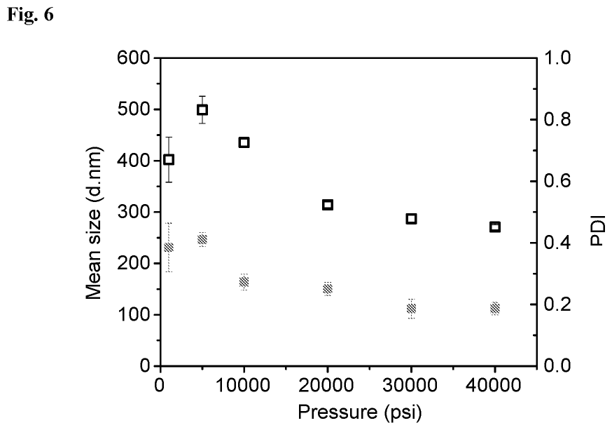

The effect of pressure on the particle size of α-La-Trp complex was investigated with an α-La to Trp ratio of 20:1 at pH 11 for 30 min. As shown in FIG. 6, clear differences were present in the suspensions formed after HPH under different pressure conditions. Differences were also observed in the appearance of the suspensions formed after HPH under different pressure. The particle size and PDI value increased as the pressure increased from 1000 to 5000 psi, and then decreased when the pressure was above 5000 psi. The particle size of α-La-Trp complexes formed under high pressure (>10000 psi) tends to become smaller and more uniform (PDI<0.3) than those formed under low pressure. While not wishing to be bound to any particular theory, this may be due to the higher shear force, cavitation, and turbulence under higher pressure, which may facilitate homogeneous kinetics during particle formation. Thus, the HPH pressure from 20000 to 40000 psi was suitable for the subsequent BBD experiments.

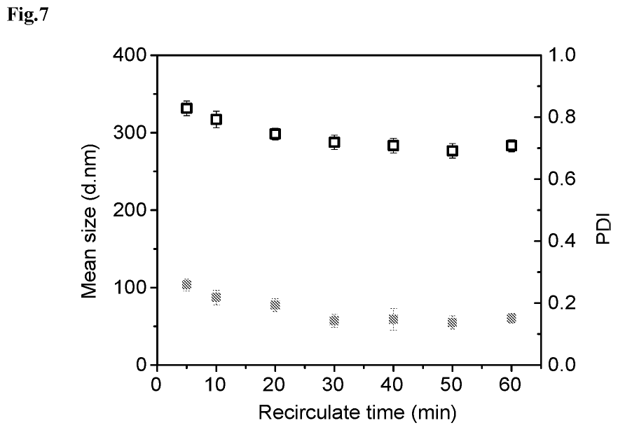

The effect of recirculate time on the particle size of α-La-Trp complex was investigated with an α-La to Trp ratio of 20:1 at pH 11 under a pressure of 30000 psi. As shown in FIG. 7, the particle size and PDI value increased as the pressure decreased from 5 to 30 min psi, and then reach a plateau of particle size of 280 nm at 30 min. Consequently, the recirculation time range of 20-40 minutes was considered for further BBD experiment.

### 3. Preparation of α-La-Trp Complex Particles Using Box-Behnken Design

Based on the above findings, a three-level, three-factor Box-Behnken design was performed to determine which combination of HPH variables would yield the smallest and stable α-La-Trp complex particles. The Box-Behnken design matrix and the mean size and PDI values under different experimental conditions are shown in Table 2. All the results were placed in Box-Behnken design of JMP to obtain the predicted values and final conclusion. All the responses of these runs significantly fitted the quadratic models (p<0.0001) with insignificant lack of fit (p>0.05). Fitting of the model to the experimental data was examined with the error analysis. As can be seen from Table 2, predicted values are closer to the experimental values. Error values are from 0.5 to 21.4 nm and from 0.001 to 0.048 for mean particle size and PDI, respectively, that is, developed models fit well with the experimental data.

TABLE 2
Box-Behnken design matrix and the experimental (Exp) and predicted (Pred) responses.

| | Levels of independent | | | pH 11 | | | | | | pH 7 | | | | | |
|---|---|---|---|---|---|---|---|---|---|---|---|---|---|---|---|
| | factors | | | Mean size (d · nm) | | | PDI | | | Mean size | | | PDI | | |
| Run | X1 | X2 | X3 | Exp | Pred | Error | Exp | Pred | Error | Exp | Pred | Error | Exp | Pred | Error |
| 1 | 15:1 | 30000 | 40 | 290.8 | 297.7 | 6.9 | 0.137 | 0.140 | 0.003 | 233.7 | 234.2 | 0.6 | 0.202 | 0.230 | 0.027 |
| 2 | 15:1 | 20000 | 30 | 361.2 | 347.5 | 13.7 | 0.183 | 0.185 | 0.002 | 300.5 | 293.9 | 6.6 | 0.246 | 0.240 | 0.006 |
| 3 | 10:1 | 40000 | 40 | 270.5 | 261.1 | 9.4 | 0.178 | 0.176 | 0.002 | 187.6 | 191.2 | 3.6 | 0.309 | 0.281 | 0.029 |
| 4 | 10:1 | 40000 | 20 | 267.7 | 260.9 | 6.8 | 0.127 | 0.132 | 0.005 | 192.2 | 186.1 | 6.1 | 0.331 | 0.352 | 0.022 |
| 5 | 15:1 | 40000 | 30 | 274.4 | 276.9 | 2.5 | 0.100 | 0.099 | 0.001 | 222.2 | 218.1 | 4.2 | 0.183 | 0.184 | 0.001 |
| 6 | 10:1 | 30000 | 30 | 307.1 | 297.4 | 9.7 | 0.183 | 0.161 | 0.022 | 209.2 | 224.5 | 15.3 | 0.343 | 0.298 | 0.045 |
| 7 | 10:1 | 30000 | 30 | 279.2 | 297.4 | 18.2 | 0.133 | 0.161 | 0.028 | 203.1 | 224.5 | 21.4 | 0.320 | 0.298 | 0.022 |
| 8 | 10:1 | 20000 | 20 | 339.2 | 329.5 | 9.7 | 0.251 | 0.253 | 0.002 | 290.3 | 286.7 | 3.6 | 0.314 | 0.342 | 0.029 |
| 9 | 5:1 | 30000 | 20 | 295.7 | 283.9 | 11.7 | 0.196 | 0.166 | 0.029 | 234.2 | 233.7 | 0.5 | 0.351 | 0.324 | 0.028 |
| 10 | 5:1 | 30000 | 40 | 276.9 | 272.6 | 4.4 | 0.178 | 0.183 | 0.005 | 223.8 | 213.6 | 10.3 | 0.228 | 0.250 | 0.022 |
| 11 | 10:1 | 20000 | 40 | 305.8 | 312.6 | 6.8 | 0.213 | 0.208 | 0.005 | 229.0 | 235.1 | 6.1 | 0.372 | 0.351 | 0.022 |
| 12 | 5:1 | 20000 | 30 | 317.4 | 314.9 | 2.5 | 0.201 | 0.202 | 0.001 | 262.3 | 266.5 | 4.1 | 0.277 | 0.276 | 0.001 |
| 13 | 15:1 | 30000 | 20 | 306.6 | 302.9 | 3.6 | 0.157 | 0.158 | 0.001 | 250.4 | 260.6 | 10.2 | 0.242 | 0.220 | 0.023 |
| 14 | 5:1 | 40000 | 30 | 251.6 | 265.3 | 13.8 | 0.137 | 0.135 | 0.003 | 191.2 | 197.8 | 6.6 | 0.266 | 0.272 | 0.006 |
| 15 | 10:1 | 30000 | 30 | 305.8 | 297.4 | 8.4 | 0.167 | 0.161 | 0.006 | 207.4 | 224.5 | 17.1 | 0.346 | 0.298 | 0.048 |

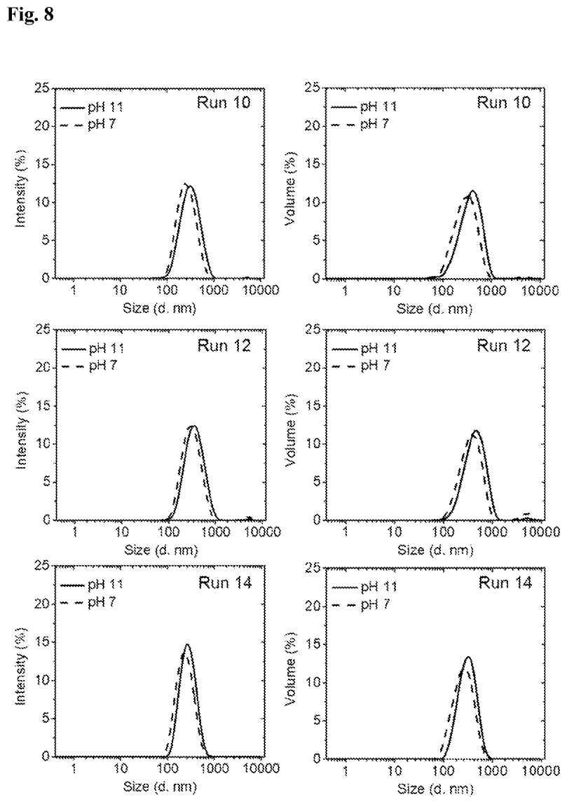

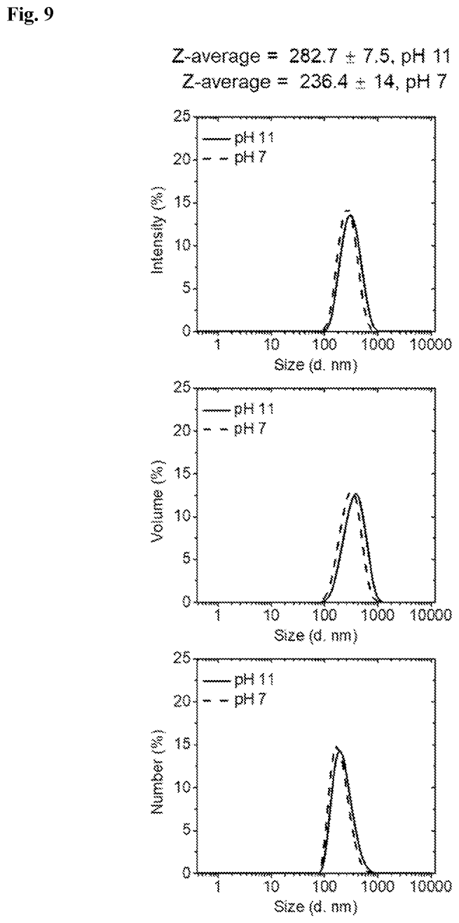

It is interesting to note that the α-La-Trp complex particles become stable as demonstrated by having one peak particle size distribution at pH 7 at α-La to Trp ratio 5:1 when using a different combination of HPH pressure and recirculation time, e.g., 30000 psi for 40 min, 20000 psi for 30 min, and 40000 psi for 30 min (see, Table 2, runs 10, 12 and 14, and FIG. 8). Considering the main purpose for encapsulating more tryptophan in the particle and the operability in the actual processing procedure, the confirmatory experiment was performed under the following conditions: α-La and Trp ratio of 5:1, under the pressure of 30000 psi for 40 min. As shown in FIG. 9, under these conditions, predicted values are closer to the experimental values. This confirms the suitability of the model for the prediction of process behavior.

### 4. Stability of α-La-Trp Complex Nanoparticles

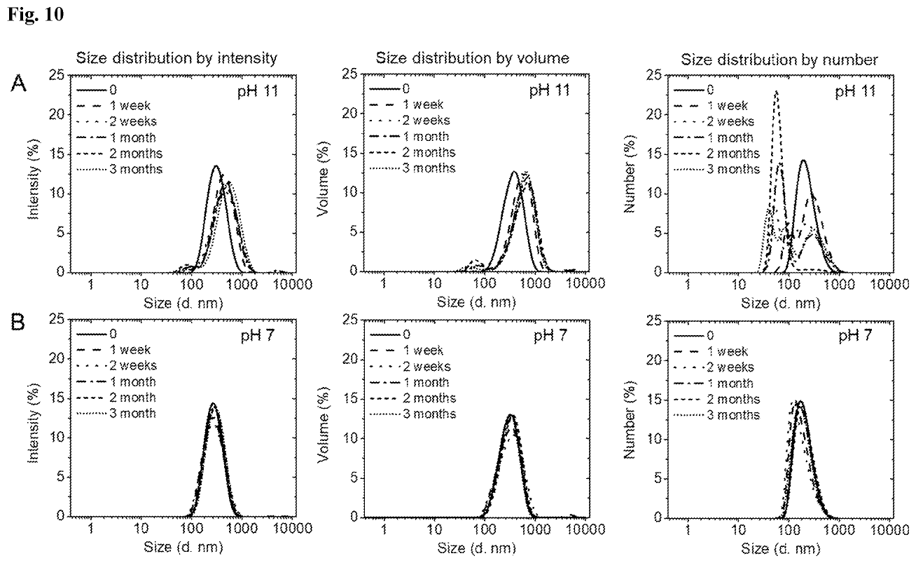

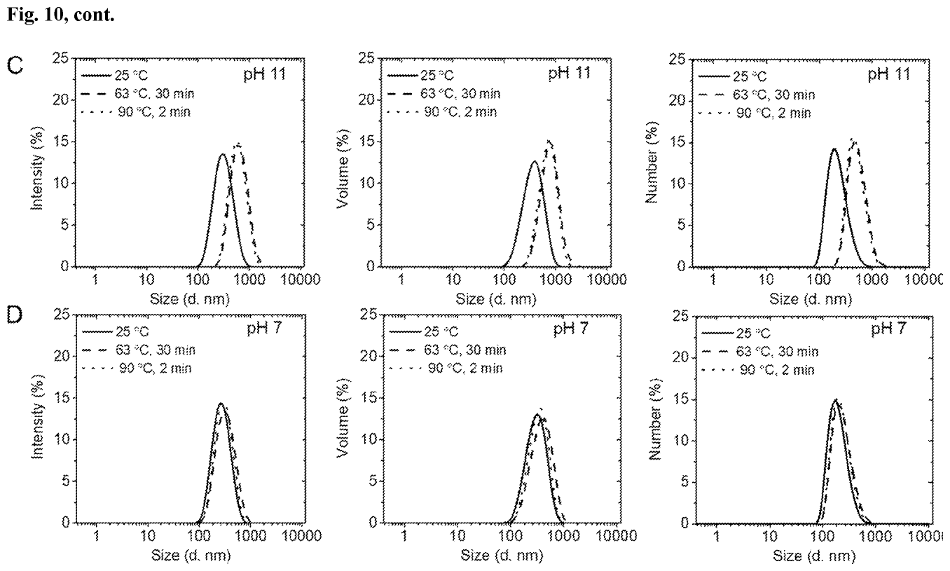

α-La-Trp complex nanoparticles were stored in a closed container at 4° C. for up to three months. The effect of storage time on size of α-La-Trp complex nanoparticles and their stability in water at pH 11 and pH 7 is presented in FIG. 10, panel A and FIG. 10, panel B. These α-La-Trp complex nanoparticles were produced by HPH at the α-La-Trp weight ratio of 5:1, 30000 psi for 40 minutes. As shown in FIG. 10, panel A, at pH 11, the particle size distribution of α-La-Trp complex nanoparticles slightly shifted to a larger size range during storage for 14 days based on the intensity and volume, while a peak for small size particles was observed by number. This may be due to the swelling and/or aggregation of the particles as well as the release of small particles during storage at pH 11. However, the particle size distribution of α-La-Trp complex nanoparticles remained stable at pH 7 (FIG. 10, panel B), where the particle size did not change significantly upon storage. While not wishing to be bound to any particular theory, this indicates that pH shifting from pH 11 to pH 7 stabilized the α-La-Trp complex nanoparticles produced by HPH.

The effect of temperature on the stability of α-La-Trp complex nanoparticles was determined after different thermal processing conditions: 63° C. for 30 min and 90° C. for 2 min. As shown in FIG. 10, panel C, after incubation at 63° C. for 30 min, the particle size of α-La-Trp complex nanoparticles at pH 11 shifted to larger size range due to the swelling and/or aggregation of the particles. Similar results were observed for samples incubated at 90° C. for 2 min. However, for the α-La-Trp complex nanoparticles at pH 7 (FIG. 10, panel D), the particle size distribution remained unchanged upon heating. This further confirms the higher stability of α-La-Trp complex nanoparticles after pH shifting (from pH 11 to pH 7).

### Example 2

Bovine milk α-lactalbumin powder was kindly provided by Agropur, USA (batch number: JE 0001-21-414, 92.5% purity). Pure tryptophan was purchased from Sigma (St. Louis, MO, USA). All water used was Milli-Q water. All other chemicals were of analytical grade.

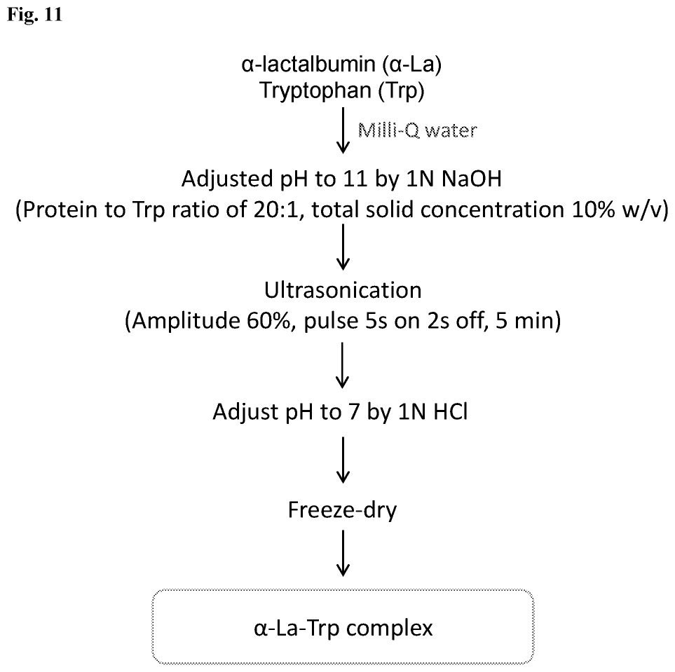

The procedure used for the α-La-Trp complex formation is shown in FIG. 11. Trp was mixed with α-La at α-La to Trp weight ratio of 20:1. The mixture of α-La and Trp was fully dissolved in Milli-Q water by adjusting pH to 11 using 1N NaOH solution (final total solid concentration of α-La and Trp was 10%, w/v). The mixture was then sonicated for 5 minutes using a 20 kHz ultrasonic processer (VibraCell, Sonics and Materials Inc.) equipped with a probe transducer and a flat tip of ½″ (13 mm). After ultrasonication, the α-La-Trp suspension was then adjusted the pH back to neutral (pH 7) using 1N HCl solution.

Upon dissolving α-La, Trp, or a mixture of α-La and Trp at pH 11, a clear solution of was observed. The solution of α-La alone remain unchanged after ultrasonication and pH shifting treatment. However, clear differences were observed in the appearance of Trp and the mixture of α-La and Trp with and without ultrasonication treatment. Trp alone and the mixture of α-La-Trp exhibited a colloidal dispersion without any visible aggregation. These colloidal dispersions remained stable in that the particles did not fall out of the composition when adjusting the pH of the composition to a pH of 7.

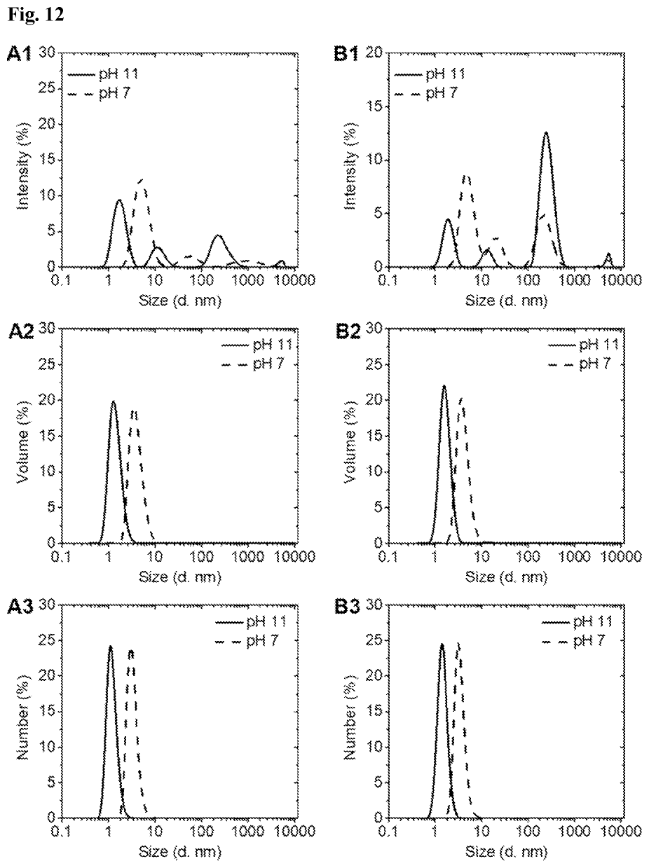

The particle size distribution of α-La before and after ultrasonication was measured by dynamic light scattering (DLS). As shown in FIG. 12, α-La are mainly small particles with a mean size of 6.5±0.1 nm at pH 11. The particle size shifts to a larger size range with a mean size of 15.5±3.6 nm when adjusting the pH from 11 to 7. After ultrasonication, both large and small particles were observed for α-La, which show a very polydisperse particle size distribution. The mean size of α-La increased to 169.8±55.8 nm. While not wishing to be bound to any particular theory, this indicates that ultrasonication may promote the association (e.g., hydrophobic interaction, electrostatic interaction, hydrogen bonding, etc.) among α-La molecules. When shifting pH back to 7, the mean particle size decreased to 41.9±42.4 nm due to the fold of the protein during pH shifting.

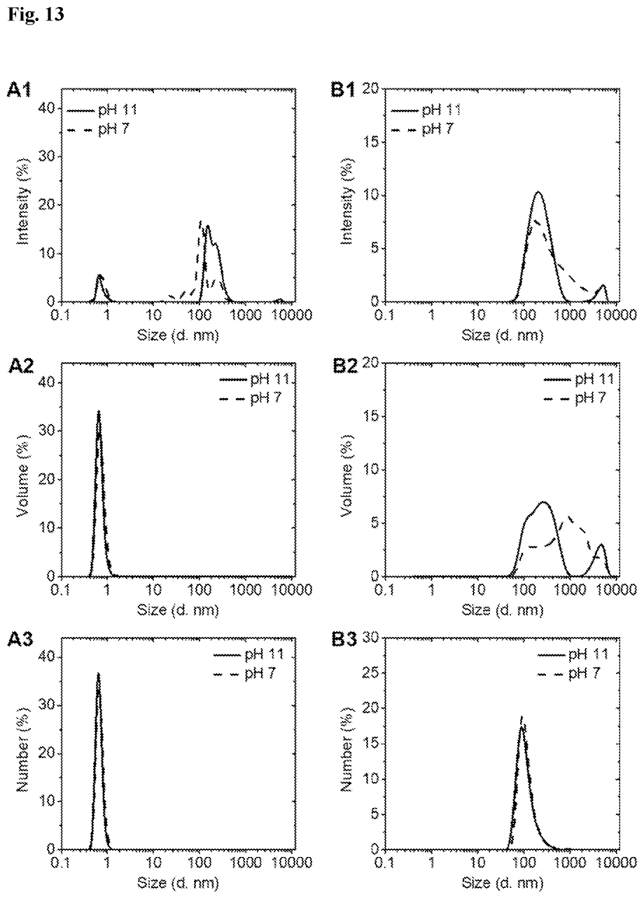

The particle size distribution of Trp before and after ultrasonication was measured by DLS. As shown in FIG. 13, Trp at pH 11 is very polydisperse with a broad particle size distribution. The particle size shifted to a larger size range when adjusting the pH from pH 11 to pH 7 (FIG. 13, panel A1). During ultrasonication, Trp molecules associate to form Trp-Trp complex particles. The particle size distribution became more uniform with a mean size of 215.1±4.4 nm (panels B1-B3 of FIG. 13). When adjusted the pH from pH 11 to pH 7, the mean size of Trp particles increased to 270±7.0 nm due to the aggregation of the particles.

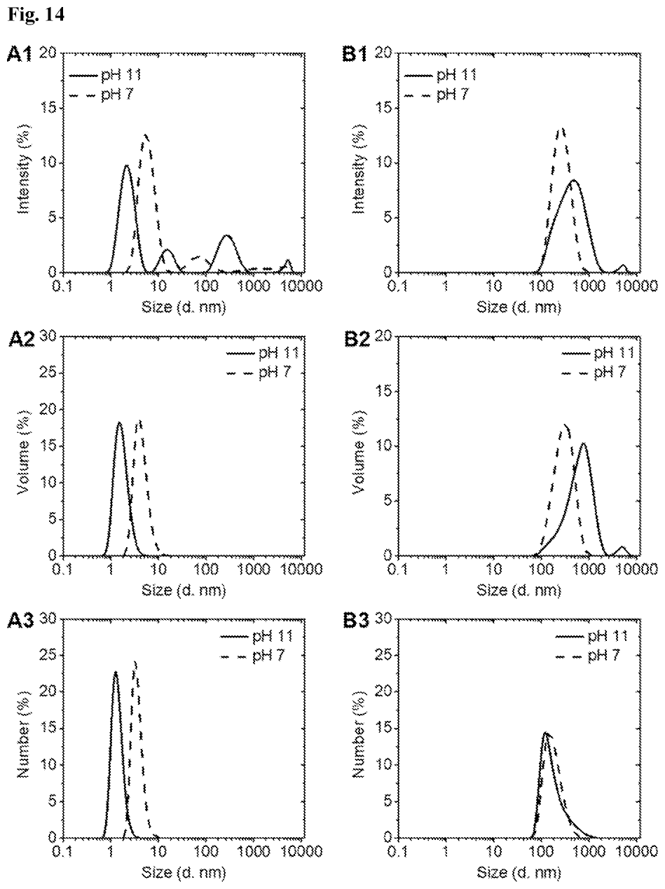

The particle size distribution of α-La-Trp before and after ultrasonication was measured by DLS. As shown in FIG. 14, the mixture of α-La and Trp at pH 11 before ultrasonication showed a similar particle size distribution as α-La with a mean size of 6.8±0.1 nm. The particle size shifted to a slightly larger size range when adjusting the pH from pH 11 to pH 7 (mean size: 7.9±0.7 nm) (FIG. 14, panel A1). During ultrasonication, α-La and Trp molecules associate to form α-La and Trp complex particles. The particle size distribution became more uniform with a mean size of 363.9±11.4 nm (FIG. 14, panels B1-B3). However, when the pH was adjusted from pH 11 to pH 7, the mean size of α-La-Trp particles decreased to 211.7±5.7 nm, which, without wishing to be bound to any particular theory, may be due to the fold of the protein molecules in the particle. While not wishing to be bound to any particular theory, the presence of Trp may promote particle formation of α-La molecules, while the presence of α-La molecules could enhance the molecular association during pH shifting due to the fold of protein molecules.

### Example 3

Freeze-dried samples of α-La-Trp particles (formed at a weight ratio (La:Trp) of 5:1 with a total solids concentration of 100 mg/mL with 30000 psi for 40 minutes) were redispersed in buffer (phosphate-buffered saline (PBS), pH7) and diluted into various concentrations (0.01, 0.05, 0.1, 0.2, 0.5, and 1.0 mg/mL) for ABTS⁺⋅ antioxidant activity assay. ABTS⁺⋅ was produced by mixing potassium persulfate (2.6 mmol/L) with ABTS solution (7.4 mmol/L) and was then kept at room temperature in the dark for 16 h. The radical solution was adjusted with phosphate-buffered saline (PBS, pH 7.4) to an absorbance of 0.70±0.02 at 734 nm. Sample with various concentrations (20 μL) were mixed with 200 μL of ABTS⁺⋅ solution at room temperature for 20 min. The absorbance was measured at 734 nm. ABTS scavenging activity was calculated using the following equation:

$$Scavenging activity ( \% ) = \frac{{A}_{0} - ( {A}_{1} - {A}_{2} )}{{A}_{0}}$$

where A₀ was the absorbance of the control group (buffer instead of the sample solution); A1 was the absorbance of the test group; and A2 was the absorbance of the sample with buffer instead of ABTS.

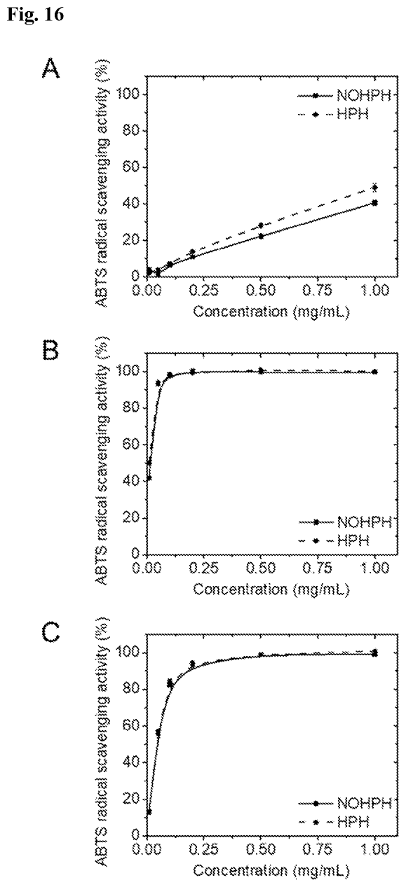

The antioxidant activity of α-lactalbumin increased after HPH treatment (panel A of FIG. 16). Tryptophan exhibited very strong antioxidant activity, which had an ABTS radical scavenging activity of around 100% with a concentration above 0.1 mg/mL (panel B of FIG. 16). At a very low concentration of 0.01 mg/mL, the antioxidant activity of tryptophan increased with HPH treatment. The ABTS radical scavenging activity of the complex nanoparticles did not change significantly compared to the mixture of α-lactalbumin-tryptophan mixture without HPH treatment (panel C of FIG. 16). However, they are substantially greater than α-La alone (panels A and C of FIG. 16). At the concentration of 0.5 mg/mL, the ABTS radical scavenging activity of the complex was around 100%, while it of α-La was 20%. Overall, tryptophan enrichment significantly increased the antioxidant activity of α-lactalbumin. HPH treatment did not significantly impact the antioxidant activity of tryptophan present in the α-La-Trp particles.

### Example 4

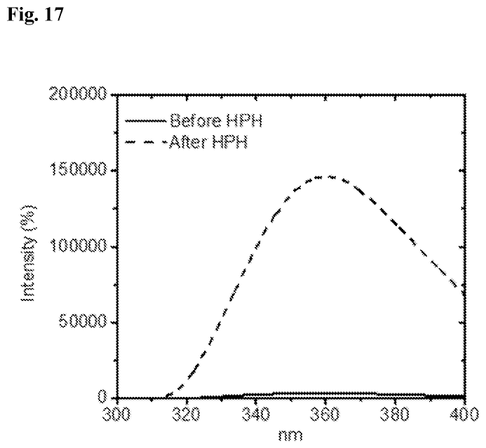

To measure the intrinsic fluorescence intensity of a mixture of α-La and Trp before and after HPH treatment, the fluorescence experiment was designed and performed. The particle formation conditions used were at a weight ratio (La:Trp) of 5:1 and a total solids concentration of 100 mg/mL with 30000 psi for 40 minutes at pH 11. The control sample was the mixture of α-La and Trp before HPH with a weight ratio (La:Trp) of 5:1 and a total solids concentration of 100 mg/mL at pH 11. The fluorescence spectra were performed by a spectrofluorometer (SHIMADZU, RF-6000, Japan) using a 1.0 cm path-length quartz cells within a thermostat bath. The excitation wavelength was set to 295 nm and the emission spectra were recorded between 310-400 nm. Data was collected at 0.5 nm wavelength resolution. As shown in FIG. 17, the protein and tryptophan molecules are closely associated after HPH with an increase in the intensity of intrinsic fluorescence.

### Example 5

The storage stability of α-La-Trp particles (formed at a weight ratio (La:Trp) of 5:1 and a total solids concentration of 100 mg/mL with 30000 psi for 40 minutes) for three months at 4° C. was examined. As shown in panels A and B of FIG. 10, the size of the particle remained stable after storage for three months at 4° C. The size change of the particles was less than 15% of its original size (increased from 223.8 nm to 249.9 nm).

### Example 6

### 1. Introduction

The application of a combined treatment of high-pressure homogenization (HPH) and pH-shifting on a mixture of α-lactalbumin (α-LA) and tryptophan (Trp) to fabricate nanoparticles (α-LA-Trp-NP) is described. Processing parameters, such as the «-LA/Trp ratio, HPH pressure, and recirculation time, were investigated to probe their effect on the physicochemical properties of α-LA-Trp-NP. The desired α-LA/Trp ratio (5:1), HPH pressure (206.8 MPa), and recirculation time (40 min) was found to produce small «-LA-Trp-NP (243.0±7.2 nm) with a narrow particle size distribution. The formation of α-LA-Trp-NPs was found to follow a controlled HPH-induced aggregation mechanism where the soluble unfolded protein and the amino acids gradually associate and grow to larger particle sizes, aggregates, that become insoluble. Comparing the size of and morphology of α-LA-NPs with α-LA-Trp-NPs indicates that the presence of Trp significantly affected the size and morphology of the NPs in the dry form. The freeze-dried α-LA-Trp-NPs could be re-dispersed easily to form a uniform nanoparticle (NP) dispersion in Milli-Q water and phosphate buffered saline (PBS) buffer. The thermal, freeze-thaw, and freeze-thaw-thermal stability of the α-LA-Trp-NPs was improved by using the combination of HPH and pH-shifting. Compared to NPs of Trp alone, the complex NPs showed better freeze-thaw stability and retained the particle characteristics with heat treatment at 63° C., 30 min after the freeze-thaw cycle. α-LA-Trp-NPs were also observed to have remarkable stability against pH changes and thermal treatments at 63° C., 30 min, and 90° C., 2 min.

### 2. Materials and Methods

### 2.1. Materials

Bovine milk α-lactalbumin (α-LA) powder was provided by Agropur, USA (batch number, JE 0001-21-414, 92.5% purity). Tryptophan (Trp) was purchased from Sigma (Reagent grade>98%, St. Louis, MO, USA). Hydrochloric acid (HCl, ACS grade) and sodium hydroxide (NaOH, 98%) were purchased from Fisher Scientific (Hampton, NH, USA). All water used was Milli-Q water. Milli-Q water (18.2 MΩ/cm) was produced using a Millipore water purification system (Millipore Sigma, Burlington, MA, USA).

### 2.2. Preparation of α-LA-Trp-NPs

α-LA-Trp-NPs were prepared using a high-pressure homogenization-pH shifting technique. Briefly, the mixtures of α-LA and Trp was fully dissolved in Milli-Q water by adjusting pH to 11 using 1 N NaOH solution. The mixture was then passed through a high pressure homogenizer (Nano DeBee, Bee International, inc. USA) and recirculated. After recirculation, the pH of the collected α-LA-Trp-NPs dispersions was adjusted to neutral (pH 7) using 1 N HCl solution. For comparison, control samples, α-LA-NPs without Trp and Trp-NPs without α-LA, were prepared using similar procedures.

### 2.3. Formulation and Process Parameters of α-LA-Trp-NPs

Formulation (α-LA to Trp ratios) and process parameters (HPH pressure and recirculation time) were investigated based on physicochemical characteristics of α-LA-Trp-NPs including particle size, particle size distribution, polydispersity index (PDI) as well as tryptophan fluorescence intensity (TFI). The solid concentration of 10 w/v % was selected after a series of preliminary studies (data not shown), where a maximum level of solid concentration with uniform particle size distribution was observed by DLS with a PDI value of less than 0.3. Different α-LA to Trp w/w % ratios (20:1, 15:1, 10:1, 5:1, 4:1, and 3:1) were tested while keeping the HPH pressure (206.8 MPa) and recirculation time (30 min) constant. α-LA-Trp-NPs were prepared at various HPH pressures (6.9, 34.5, 68.9, 137.9, 206.8, and 275.8 MPa) which were systematically changed while mass ratio and recirculation time (30 min) were kept constant. Finally, the effect of recirculation time (5, 10, 20, 30, 40, 50, and 60 min) was explored by keeping the HPH pressure (206.8 MPa) and mass ratio constant.

### 2.4. Measurements of Particle Size, Particle Size Distribution, and Polydispersity Index

The particle size, particle size distribution, and PDI values of the NPs were determined by dynamic light scattering (DLS) using a Zetasizer (Nano S, Malvern Instruments, Worcestershire, UK). The measurements were performed at a scattering angle of 173°, at 25° C. and 15 runs were done for each measurement. Particle size distribution can also be reflected by PDI values ranging between 0 and 1; a small PDI value indicates a narrow size distribution. In most cases, a monodispersed particle system would have a PDI less than 0.3. All measurements were carried out in triplicate.

### 2.5. Turbidity Measurements

The turbidity of the α-LA-Trp mixtures was measured using a UV-Vis light spectrophotometer (UV-2600, SHIMADZU Co., Japan). Sample solutions were analyzed at room temperature and the transmittance was measured at 600 nm. Milli-Q water was used as the blank reference (100% transmittance). The turbidity (T) was calculated as follows (Eq. 1):

$$T = - ln \frac{I}{I 0} \qquad ( 1 )$$

where I is the transmittance intensity of the sample solution and I0 is the transmittance intensity of the blank.

### 2.6. Tryptophan Fluorescence Spectra Analysis

The tryptophan fluorescence spectra of α-LA and α-LA-Trp mixtures were performed using a spectrofluorometer (SHIMADZU, RF-6000, Japan) according to a previous method with some modifications (Zhu et al., (2021) Physicochemical and functional properties of a novel xanthan gum-lysozyme nanoparticle material prepared by high pressure homogenization. LWT, 143, 111136.). Briefly, the fluorescence excitation wavelength was set at 295 nm, and the emission was recorded in a range between 310-500 nm. Data were collected at a step resolution of 0.5 nm. The change in fluorescence intensity of the protein and its combination with tryptophan were monitored during nanoparticle formation, including the fluorescence intensity of α-LA-Trp mixtures under different HPH conditions and that of α-LA, Trp and α-LATrp mixtures at pH 11 before and after HPH as well as those after adjusting pH back to 7.

### 2.7. Scanning Electron Microscopy (SEM)

The morphology of α-LA, Trp and α-LA-Trp mixtures with and without HPH were observed using an SEM (Zeiss Gemini 500, Jena, Germany). Freeze-dried samples were directly mounted on an SEM plate with a carbon conductive tab before coating, while liquid samples (without freeze-drying) were diluted with PBS (10 mM, pH 7) to a solid concentration of 1 mg/mL and dripped onto an SEM plate with carbon conductive tab and allowed to vacuum dry at room temperature. All samples were coated with carbon using a sputter coater (Denton Desk V, New Jersey, USA) and were then scanned by SEM with 1 keV and imaged by a high-efficiency secondary electron detector with a 20.0 μm aperture.

### 2.8. Stability Analysis

### 2.8.1 pH Stability

The effect of HPH treatment and pH shifting on the pH stability of α-LA, Trp, and mixtures of α-LA and Trp was investigated and compared. The pH of all dispersions, with and without HPH, at pH 11 was adjusted to pH 7 and 3 by adding 1.0 M HCl. The appearance, turbidity, particle size, particle size distribution, and PDI were measured and recorded.

### 2.8.2 Thermal Stability

The effect of HPH treatment and pH shifting on the thermal stability of α-LA, Trp, and the α-LA-Trp mixture was investigated and compared. Samples, with and without HPH, at pH 11 and those after pH shifting at pH 7 and 3 were incubated at 63° C. for 30 min (pasteurization), and 90° C. for 2 min (flash pasteurization), and then cooled to 25° C. The appearance, turbidity, particle size, particle size distribution, and PDI were measured and recorded.

### 2.8.3 Freeze-Thaw and Freeze-Thaw-Thermal Stability

The effect of HPH treatment and pH shifting on the freeze-thaw and freeze-thaw-thermal stability of α-LA, Trp, and mixtures of α-LA and Trp was investigated and compared. Samples, with and without HPH, at pH 11 and those after pH shifting at pH 7 were stored in a freezer at −20° C. overnight and then transferred to a water bath with a temperature set at 25° C. for 2 h until completely thawed. The appearance, turbidity, particle size, and particle size distribution were recorded after the freeze-thaw cycle. The freeze-thaw samples were further heated at 63° C. for 30 min to investigate the freeze-thaw-thermal stability.

### 2.9. Redispersion of the Freeze-Dried NPs

Freeze-dried α-LA-NPs, Trp-NPs, and α-LA-Trp-NPs (10 mg) were redispersed in 10 mL Milli-Q water or PBS buffer (10 mM, pH7) and stirred for 2 h. The particle size, particle size distribution, and PDI of the NPs in the dispersion were measured by DLS.

### 2.10. Statistical Analysis

All experiments were performed in triplicate. Results are presented as mean±SD (standard deviation). The significance between means was established using one-way ANOVA (OriginPro 9.0.0, OriginLab Northampton, MA, USA).

JMP Pro 16.0.0 software (SAS Institute Inc., Cary, NC, USA) was used to develop the experimental design and to analyze the data. To perform sensitivity analysis of processing parameters, we adopted the Design of Experiment (DOE) approach. For this study, a Full Factorial Design (3K) strategy was selected, and two variables (K=2) were analyzed: HPH pressure (X1, in MPa) and recirculation time (X2, in min). The investigated responses were particle size (Y1) and TFI (Y2) at pH 11, particle size (Y3), and TFI (Y4) at pH 7.

### 3. Results and Discussion

HPH and pH shifting were used to form nanoparticles containing α-LA and Trp. Without wishing to be bound to any particular theory, results indicate that at high pH, the protein was unfolded offering open sites and areas for the incorporation of Trp. HPH mixed the protein and the Trp molecules together to form nanoparticles. The pH of the NP solutions was then lowered and believed to allow the proteins to fold around the Trp, creating smaller NP with higher Trp fluorescence.

### 3.1. The Effect of HPH on the Formation of α-LA-Trp-NPs

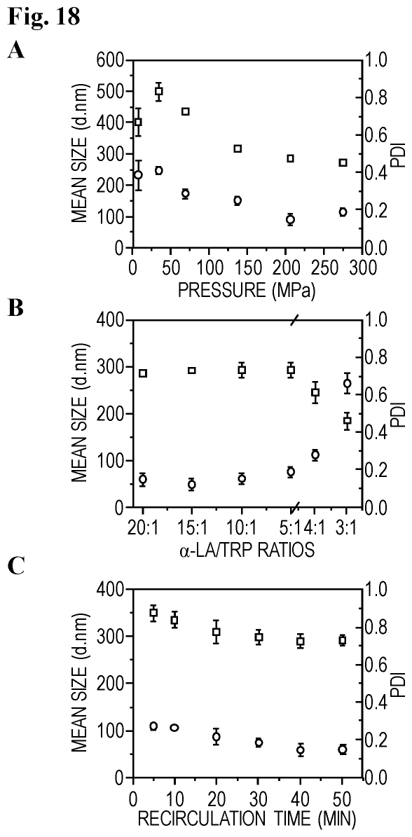

We used screening tests to probe the effects of HPH on the hydrodynamic particle size of the α-LA-Trp-NPs, the HPH pressure and recirculation time, as well as varied α-LA/Trp ratios. The mean particle size (open square) and PDI values (polydisperse index, solid square) of α-LA-Trp-NPs prepared at different conditions were measured using DLS (FIG. 18). The effect of pressure on the particle size of α-LA-Trp-NPs was investigated with a α-LA to Trp ratio of 20:1 (w/w) at pH 11 for 30 min (Panel A of FIG. 18). The particle size and PDI value of α-LA-Trp-NPs increases as the pressure increases from 6.9 to 34.5 MPa, and then decreases when the pressure was above 34.5 MPa (Panel A of FIG. 18). Generally, the particle size of α-LA-Trp-NPs formed under high pressure (>68.9 MPa) decreased and became more uniform (PDI<0.3) than those formed under low pressure. This is possibly due to the higher shear force, cavitation, and turbulence under high pressure, which facilitates protein aggregation and the formation of colloidal particles derived from protein aggregation. The mean particle size of α-LA-Trp-NPs formed was slightly decreased from 436 to 271 nm with increasing HPH pressure from 68.9 to 275.8 MPa. PDI decreased from 0.29 at 68.9 MPa to 0.15 at 206.8 MPa, but then slightly increased to 0.19 at 275.8 MPa. Without wishing to be bound to any particular theory, this may be attributed to the particles becoming increasingly polydisperse with increasing HPH pressure, and greater intermolecular interactions may become available at lower pressures which break down at higher pressures.

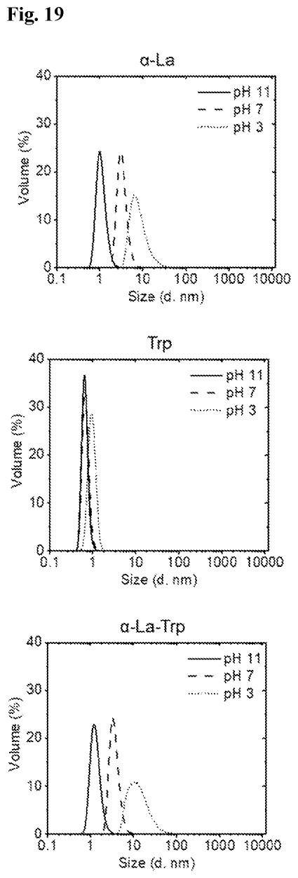

The effect of α-LA/Trp ratios (20:1, 15:1, 10:1, 5:1, 4:1, and 3:1 w/w) on the particle size of α-LA-Trp-NPs was performed under a HPH pressure of 206.8 MPa for a recirculation time of 30 min. Between the range of 20:1 and 5:1 α-LA to Trp ratios, we observed no significant changes in the particle size and PDI values, these NPs showed a narrow particle size distribution with a mean particle size ˜290 nm (Panel B of FIG. 18). When the Trp content was further increased to a α-LA/Trp ratio of 4:1 and 3:1, the particle size decreased significantly and were not uniform (PDI>0.3). Therefore, the α-LA/Trp ratio of 5:1 was selected for further study. The particle size and PDI values of La-Trp-NPs decreased with increasing recirculation time from 5 to 30 min, and then reached a plateau of mean particle size of ˜290 nm from 30 min (Panel C of FIG. 18). Comparing the particle size of α-LA-Trp mixture (α-LA/Trp of 5:1, w/w) that was not treated with HPH (NOHPH) at pH 11 (FIG. 19), the particle size of La-Trp-NPs increased from 7 nm with PDI value of 0.37 (FIG. 19) to 332 nm with PDI value of 0.26 after 5 min HPH. This result indicated that HPH may induce the aggregation of α-LA and Trp molecules forming large particles within 5 min under HPH at a pressure of 206.8 MPa. This is in agreement with visual differences observed in turbidity from the appearance of colloidal dispersions formed after HPH under different recirculation times.

### 3.2. Detection of Aggregation Via Tryptophan Fluorescence

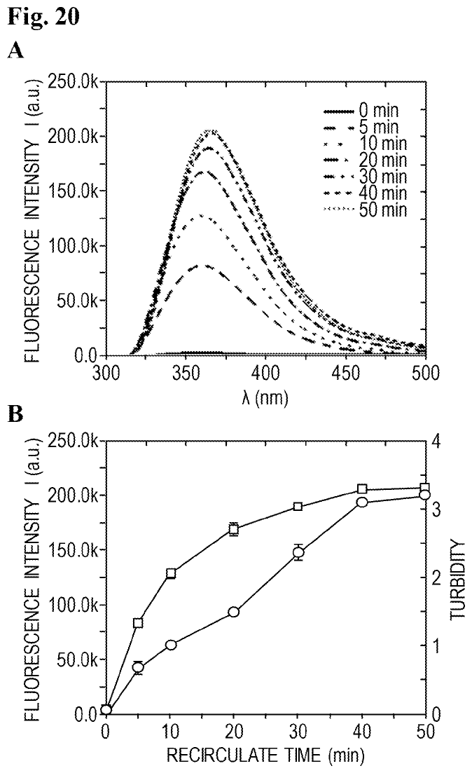

The aggregation mechanism of the α-LA-Trp mixture was studied by monitoring the change in both the turbidity and the Trp fluorescence change during HPH. Trp fluorescence is linked to aggregation; increasing aggregation of Trp molecules causes the Trp chromophore to increase or turn on, while at lower aggregation, the Trp turns off or exhibits lower fluorescence (Liu, Wolstenholme, et al., 2018). Initially, the α-LA-Trp mixture (α-LA:Trp of 5:1, w/w) at pH 11 without HPH showed weak fluorescence emission due to the pH dependent unfolding of the protein (Panel A of FIG. 20). However, after 50 min of HPH treatment at pH 11, the fluorescence intensity increased by ˜35-fold. The fluorescence intensity was positively correlated to the turbidity of α-LA-Trp aggregates (Panel B of FIG. 20), indicating that HPH may induce aggregation between Trp and α-LA. Therefore, we were able to use fluorescence to monitor the aggregation of α-LA-Trp and select desired NP formation conditions (see Section 3). The mechanism of NPs formation by HPH and pH shifting monitored by Trp fluorescence was further investigated and is discussed in Section 3.4.

### 3.3. Investigation of HPH Conditions

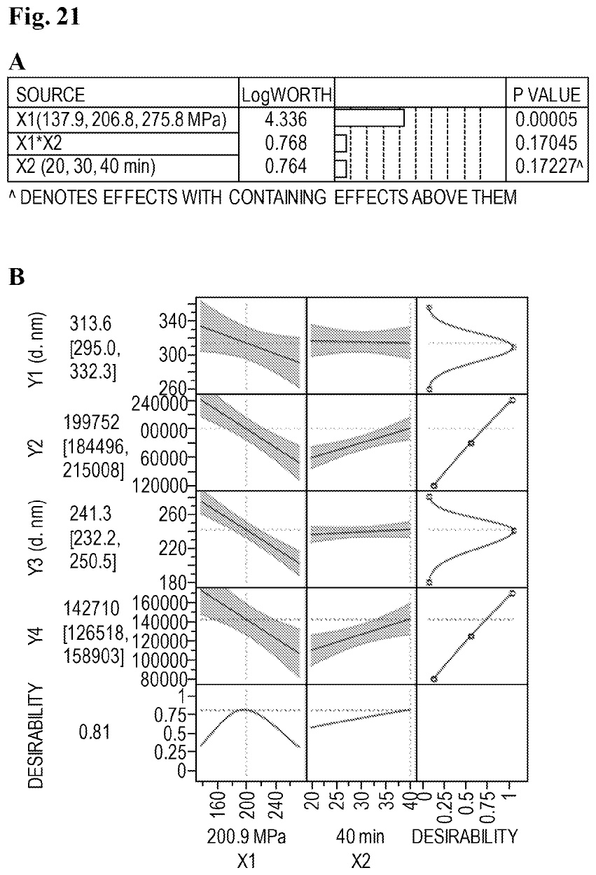

Investigation of HPH conditions with the goal of producing uniform α-LA-Trp-NPs with a small particle size and a «-LA/Trp ratio of 5:1 was carried out at pH 11 and pH 7. Particle size, PDI, and Trp Fluorescence Intensity (TFI) at pH 11 and pH 7 were measured for 9 treatment combinations of HPH pressure (137.9, 206.8, and 275.8 MPa), and recirculation time (20, 30, and 40 min) (Table 3). These 9 treatments represented the Full Factorial Design (FFD) as determined using the Design of Experiments function in the JMP software. Based on the results from our ANOVA analysis, all models have correlation coefficients (R2) in the range of 0.87 and 0.97 with p-values between 0.0003 and 0.0108 (p<0.05), which correlated well with the actual experimental data. The results indicated that particle size and TFI, at both pH 11 and shifting pH from pH 11 to pH 7 (pH 11→7), are significantly affected by the HPH pressure (p<0.05) but HPH and recirculation time did not significantly influence each other t (p>0.05) (Table 3 and Panel A of FIG. 21). Our goal was to minimize nanoparticle size with uniform particle size distribution (minimize PDI) and increase TFI at both pH 11 and pH 7. Based on these criteria, the predicted desirable conditions were: HRH pressure 200.9 MPa (29,138 PSI), and HPH time 40 min (Panel B of FIG. 21). These conditions had the highest desirability value of 0.81 (Panel B of FIG. 21). Constrained by instrument settings, we used an HPH pressure of 206.8 MPa (30,000 PSI) and a recirculation time 40 min.

TABLE 3
Experimental factorial design experiments for
α-LA-Trp-NPs in case of fixed α-LA/T

| | Factors | | pH 11 | | | pH 7 | | |
|---|---|---|---|---|---|---|---|---|
| | X₁ | X₂ | Y₁ | | Y₂ | Y₃ | | Y₄ |
| | Pressure | Time | Size | | TFI | Size | | TFI |
| Run* | (MPa) | (min) | (d · nm) | PDI | (×10³) | (d · nm) | PDI | (×10³) |
| 1 | 206.8 | 20 | 310.5 | 0.220 | 169 | 235.9 | 0.281 | 120 |
| 2 | 137.9 | 20 | 358.4 | 0.257 | 175 | 266.7 | 0.344 | 124 |
| 3 | 275.8 | 40 | 298.8 | 0.188 | 148 | 205.3 | 0.306 | 101 |
| 4 | 275.8 | 30 | 290.3 | 0.171 | 133 | 187.9 | 0.316 | 88 |
| 5 | 137.9 | 40 | 346.0 | 0.226 | 235 | 274.7 | 0.265 | 164 |
| 6 | 137.9 | 30 | 345.4 | 0.235 | 208 | 267.0 | 0.276 | 151 |
| 7 | 275.8 | 20 | 269.3 | 0.193 | 124 | 199.2 | 0.314 | 84 |
| 8 | 206.8 | 40 | 291.4 | 0.151 | 203 | 239.0 | 0.196 | 158 |
| 9 | 206.8 | 30 | 299.2 | 0.187 | 190 | 242.2 | 0.217 | 124 |

*PDI: polydispersity index; TFI: Tryptophan fluorescence intensity

The mean particle size of α-LA-Trp-NPs obtained under our desired conditions was 291 nm at pH 11 and 239 nm at pH 7. This result indicated the particles formed at pH 11 remain uniform when pH shifted to neutral conditions. The slight decrease in particle size with pH shifting may be attributed to the molecular associations, such as hydrophobic interaction, electrostatic interaction, hydrogen bonding, which are enhanced during the pH shifting, resulting in a more highly packed structure where protein molecules exhibited a higher degree of folding in the nanoparticles at pH 7. In addition, changes in the pH of the solution changed the environment of α-LA-Trp-NPs, which influenced the particle size of α-LA-Trp-NPs. This is consistent with the TFI data, where TFI decreased after shifting to pH 7; because of the increased protein molecular interactions, the Trp self-aggregation was reduced at pH 7 and the TFI was subsequently reduced. Overall, the particles formed by HPH were stable with uniform particle size distribution at both pH 11 and 7. It was noted that HPH pressure and time significantly influences (p<0.05) the particle size and PDI value of α-LA-Trp-NPs.

Finally, the conditions HPH under 206.8 MPa for 40 min were used in all the following experiments where α-LA-NPs and Trp-NPs were prepared for comparison. The mean size (Z-average) and PDI values of α-LA-NPs, Trp-NPs, and α-LA-Trp-NPs in dispersions obtained under the desired conditions are shown in Table 4. Under these conditions, the particle size of a-LA-Trp-NPs was 283.0±6.9 nm and 243.0±7.2 nm with narrow particle size distribution (PDI<0.2) at pH 11 and 7, respectively, which was consistent with the predicted results (Panel B of FIG. 21). It is interesting to note that NPs were also formed from α-LA and Trp individually under the same conditions. The particle size of the α-LA-NPs were similar to that of α-LA-Trp-NPs, while Trp-NPs showed smaller particle size around 210 nm at both pH 11 and 7. This is mainly due to the self-assembly behavior of both «-LA and Trp.

TABLE 4
Effect of pH values on the turbidity, mean particle
diameter (Z-average, d · nm) polydispersity index
(PDI) of α-LA-Trp-NPs formed under desired conditions
(HPH under 206.8 MPa for 40 min) and their α-LA-
NPs, Trp-NPs counterparts formed under the same conditions

| | | | Z-average | |
|---|---|---|---|---|
| Samples * | pH | Turbidity | (d · nm) | PDI |
| α-LA-NPs | 11 | 3.12 ± 0.28 ^{a} | 289.0 ± 3.9 ^{a} | 0.14 ± 0.02 ^{b} |
| | 7 | 3.06 ± 0.26 ^{a} | 233.0 ± 12.6 ^{c} | 0.19 ± 0.02 ^{a} |
| | 3 | 2.88 ± 0.24 ^{a} | 258.1 ± 3.2 ^{b} | 0.21 ± 0.01 ^{b} |
| Trp-NPs | 11 | 3.46 ± 0.19 ^{a} | 210.0 ± 9.4 ^{a} | 0.16 ± 0.02 ^{ab} |
| | 7 | 3.27 ± 0.18 ^{a} | 207.4 ± 4.8 ^{a} | 0.16 ± 0.01 ^{b} |
| | 3 | 3.24 ± 0.18 ^{a} | 205.7 ± 5.2 ^{a} | 0.20 ± 0.02 ^{a} |
| α-LA-Trp-NPs | 11 | 2.87 ± 0.26 ^{a} | 283.0 ± 6.9 ^{a} | 0.16 ± 0.03 ^{b} |
| | 7 | 2.69 ± 0.26 ^{a} | 243.0 ± 7.2 ^{b} | 0.18 ± 0.04 ^{b} |
| | 3 | 2.55 ± 0.23 ^{a} | 232.2 ± 13.0 ^{b} | 0.45 ± 0.11 ^{a} |

Data are means ± standard deviation. Means within columns among the same sample not sharing a common letter are significantly different at p < 0.05.
* α-LA (α-lactalbumin), Trp (tryptophan), and mixture of α-LA and Trp aggregation during NP formation.

α-LA, Trp, and a mixture of α-LA and Trp were suspended in water and dissolved by adjusting the pH to 11. At high pH the intermolecular attractions of α-LA such as disulfide bonds, hydrophobic interactions, and electrostatic interactions, are reduced and its solubility increases. By decreasing the intermolecular attractions in α-LA, the possibility for the formation of new intramolecular interactions, bonds, and structures between the protein and Trp increases. Once the solutions were dissolved, we evaluated the effect of pH shifting with and without HPH.

Shifting pH from pH 11 to pH 7 without HPH did not change the appearance of the solutions of α-LA, Trp, and the mixture of α-LA and Trp; they all remained clear solutions. The particle size of α-LA and the α-LA-Trp mixture without HPH (NOHPH) slightly increased during pH shifting which is related to the formation of soluble aggregates with a size range of 2-10 nm (FIG. 19). The presence of Trp in α-LA slightly increased the particle size of α-LA at both pH 11 and 7, indicating the complex formation between &-LA-Trp.

When HPH was applied to solutions of α-LA, Trp, and α-LA-Trp at pH 11, the samples were colloidal, insoluble dispersions. The mean particle size increased from ˜2 nm under NOHDH (FIG. 19) to ˜280 nm with HDH (Table 4), suggesting the aggregation of α-LA, Trp, and the a-LA-Trp complex to form larger sized particles under high pressure, high shear, and cavitation. The NPs formed were maintained after shifting pH from 11 to 7, but the particle size was smaller which may be attributed to protein contraction at lower pH. Without wishing to be bound to any particular theory, we concluded that HPH plays an important role in the formation of the α-LA-Trp-NPs and that while the NPs are robust under pH shifting, there is a small decrease in size.

### 3.4. Potential Mechanism of NPs Formation by HPH and pH Shifting

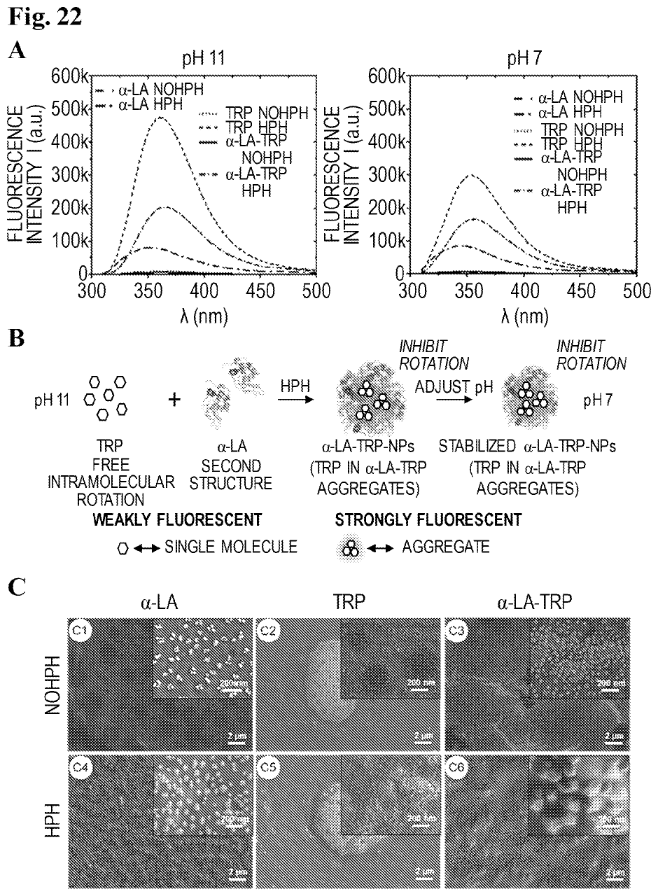

Panel A of FIG. 22 shows fluorescence spectroscopy of the α-LA, Trp, and α-LA-Trp mixture with and without HPH treatment at pH 11 and pH 7. The excitation wavelength was 295 nm. The emission peaks of the α-LA-Trp (5:1 w/w) mixture were observed in the wavelength range of 320-500 nm (Panel A of FIG. 22). At pH 11 without HPH, the α-LA, Trp, and α-LA-Trp mixtures exhibit weak fluorescence emission at pH 11. However, when HPH is applied, the emission intensity of the same samples prepared at pH 11 increased about 50-fold for α-LA, 45-fold for Trp, and 35-fold α-LA-Trp, respectively. Therefore, we speculated that HPH increased aggregation which may trigger the turn on mechanism for the Trp chromophore for all samples.

When the pH was adjusted from pH 11 to pH 7, the results remained consistent: NOHPH samples were weakly fluorescent and HPH samples showed significantly higher fluorescence intensity at pH 7 (Panel A of FIG. 22). This is consistent with the visual appearance of the samples where NOHPH samples were clear molecular solutions, while after HPH the cloudy colloidal dispersions were indicative of aggregation. This is also in agreement with the particle size results where submicron sized particles were observed for samples treated with HPH. According to previous reports (Stănciuc et al., 2012; Toptygin et al., (2002). Effect of the Solvent Refractive Index on the Excited-State Lifetime of a Single Tryptophan Residue in a Protein. The Journal of Physical Chemistry B, 106 (14), 3724-3734; Vivian & Callis, (2001). Mechanisms of Tryptophan Fluorescence Shifts in Proteins. Biophysical Journal, 80 (5), 2093-2109), exposure of internal tryptophan residues to the protein surface will result in a decrease of the fluorescence intensity due the solvent quenching. Buried tryptophan residue, however, will be less affected by the presence of the solvent and thus will show strong fluorescence. Second, previous studies on the fluorescence of the Trp chromophore indicate that free Trp molecules are mostly nonfluorescent due to rapid nonradiative decay via twisted-intramolecular charge transfer (TICT) which is blocked when the molecules are locked in place by proteins, protein aggregates, and self-assemblies causing high fluorescence (Carayon et al., (2016). Conjugates of Benzoxazole and GFP Chromophore with Aggregation-Induced Enhanced Emission: Influence of the Chain Length on the Formation of Particles and on the Dye Uptake by Living Cells. Small, 12 (47), 6602-6612; Liu, et al. (2018). Modulation of Fluorescent Protein Chromophores To Detect Protein Aggregation with Turn-On Fluorescence. Journal of the American Chemical Society, 140 (24), 7381-7384). Therefore, without wishing to be bound to any particular theory, we propose that α-LA-Trp-NPs formed by HPH may have Trp molecules buried deeply within the protein structure as aggregates, which would reduce the impacts of solvent quenching and inhibit TICT, thus resulting in higher fluorescence (Panel B of FIG. 22).

The slight decrease observed in the fluorescence maximum emission intensity and wavelength of α-LA-Trp mixtures due to pH shifting from 11 to 7 may be due to differences in their molecule/aggregate structure. The particle size decreased after pH shifting from ˜283 nm at pH 11 to ˜243 nm at pH 7 (Table 4). This means that more of the Trp in the mixture could be exposed to the solvent and thus the fluorescence suffered from solvent induced quenching. Studies of pH-dependent disassembly and reassembly of proteins have proposed that at higher pH, proteins unfold and disassemble, then when the pH is shifted to lower pH, they refold and reassemble at neutral pH (Tang, 2020; Tang et al., 2009). Therefore, without wishing to be bound to any particular theory, we propose that as the pH shifts from 11 to 7, the α-LA-Trp-NPs contract and we observe a decrease in fluorescence intensity due to solvent quenching of Trp that is closer to the surface of the protein (Panel B of FIG. 22).

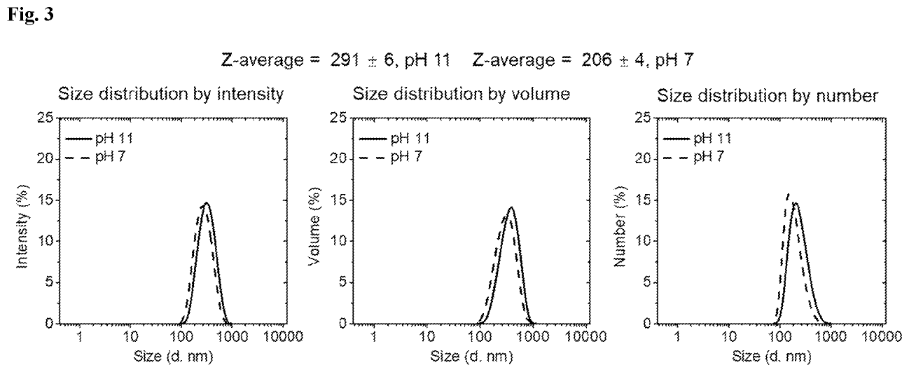

The SEM micrograph of NOHPH and HPH samples for α-LA, Trp, and α-LA-Trp dispersion (1 mg/mL) illustrated the differences observed in the morphology of the samples (Panel C of FIG. 22). The NOHPH samples showed small size aggregates, while larger size aggregates were observed for HPH samples. As shown in FIG. 3C1, The NOHPH sample of α-LA exhibited globular particles with size of ˜40 nm (Panel C of FIG. 22), while the particle of its HPH sample was larger with size of ˜80 nm (Panel C of FIG. 22). The particle size in the dry state was much smaller than the hydrodynamic particle size measured by DLS as presented in Table 2 due to the dehydration of the particle and different measurement techniques used. Similar observations were found for NOHPH and HPH samples of Trp and α-LA-Trp mixture; the relative size of the HPH samples to the NOHPH samples was consistently larger. The mixture of α-LA-Trp, however, showed different morphology than either that of α-LA or Trp; the structures appeared more crystalline and had less globular shapes. The HPH sample of α-LA-Trp showed homogenous polygonal particles with a uniform size of ˜180 nm. These particles were very different both in morphology and size when compared to α-LA and Trp. This is further evidence of complex formation between α-LA and Trp and is consistent with the results of Trp fluorescence.

### 3.5. The Colloidal Stability of α-LA-Trp-NPs at Different pH

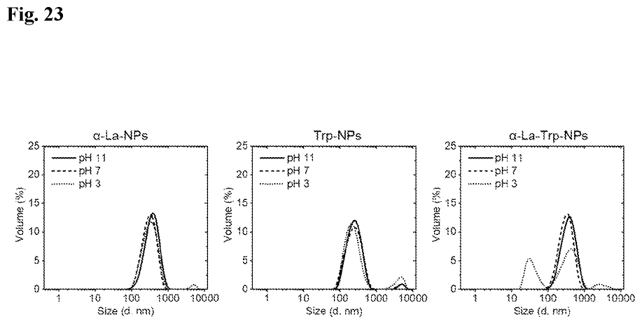

The dispersion stability of NOHPH and HPH samples after pH shifting from 11 to neutral (pH 7) and high acidic condition (pH 3) was studied by visual observation and particle size measurements (FIG. 23 and Table 4). For NOHPH samples, pH shifting did not change the appearance of α-LA, Trp, and the mixture of α-LA-Trp, which were all clear solutions at pH 11, 7 and 3.

For HPH samples, visually, the colloidal dispersion remained unchanged during pH shifting from 11 to 7 or pH 11 to 3. The appearance and turbidity of the colloidal dispersion did not change significantly (Table 4). This is consistent with the particle size results (FIG. 23), where all NPs showed larger size particles compared to those NOHPH samples. The pH stability of all NPs at pH 11, 7, and 3 indicated that the molecular interaction within the NPs induced by HPH was stable at those pH values. The PDI value of the α-LA-Trp-NPs at pH 3 was significantly increased, and a peak for small-size particles ranging from 20-100 nm was observed. The smaller particles implies that the particles were partially broken down during pH shifting from 11 to 3. Compared to the NPs formed from α-LA and Trp alone, the NPs formed from their mixture (α-LA-Trp-NPs) were less stable at pH 3. These results indicated the interaction induced by the aggregation of α-LA and Trp alone showed better stability at pH 3 than those of their mixture, which resulted in the slight release of small particles from the large particles. Overall, the NPs formed by HPH were stable in a wide pH range.

### 3.6. Effect of HPH and pH Shifting on the Thermal Stability of α-LA-Trp-NPs

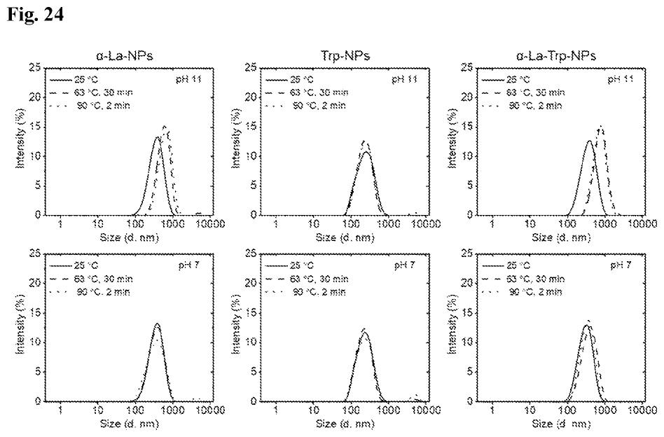

To assess the thermal stability of the HPH self-assembled α-LA-NPs, Trp-NPs, and the mixed α-LA-Trp-NPs, all solutions were incubated at 63° C. for 30 min and at 90° C. for 2 min to approximate food processing conditions. Their appearance, turbidity, particle size, and particle size distribution were determined. The thermal treatments did not impact the appearance of the colloidal dispersion of α-LA-Trp-NPs and their α-LA-NPs and Trp-NPs counterparts at both pH 11 and 7. The solution's physical appearance is consistent with the lack of change in the measured turbidity (Table 5). We speculated that the particles formed by HPH may remain intact during heat processing and this was supported by the results of particle size measurements (FIG. 24 and Table 5); the particle size distribution of all the samples after heat treatments ranged from 70 nm to 3000 nm, which is greater than the particle size of α-LA and Trp in molecular form (<10 nm, FIG. 19). This suggested the inter- or intramolecular associations induced by HPH may be maintained upon heating. The mean particle sizes (Z-average) of α-LA-Trp-NPs at pH 11 increased from ˜290 nm to ˜540 nm (63° C. for 30 min) and ˜520 nm (90° C. for 2 min). However, the mean particle size of α-LA-Trp-NPs with pH shifting (at pH 7) did not change after incubation at 63° C. for 30 min (240 to 260 nm) and at 90° C. for 2 min (240 nm to 250 nm) (Table 5). The PDI values of α-LA-Trp-NPs did not change significantly upon heating at pH 11 (PDI<0.2) indicating that the particles in the dispersion are uniform with narrow particle size distribution (FIG. 24). The growth of the particle size after heating may be attributed to the swelling of the particles. The increase in particle size of α-LA-Trp-NPs at pH 11 after heat treatments was greater than those of α-LA-NPs. For example, the particle size of α-LA-Trp-NPs increased from ˜283 to 540 nm upon heating at 63° C., 30 min, while that of α-LANPs increased from ˜289 to ˜450 nm. This may be due to the presence of Trp in the NPs, which is complex with protein and resulted in less protein-protein interaction during HPH at pH 11, promoting the swelling of α-LA-Trp-NPs upon heating. However, the particle size of Trp-NPs did not change significantly upon heating at both pH 11 and 7. This may be due to stronger intermolecular forces, such as π-π stacking, between Trp molecules, which would require more energy to overcome to allow swelling. The interesting fact is that there was no significant change in the particle size and particle size distribution of α-LA-NPs and α-LA-Trp-NPs with pH shifting from 11 to 7 upon heating (Table 5 and FIG. 24). This characteristic of α-LA-NPs and α-LA-Trp-NPs at pH 7 may be attributed to certain inter- or intramolecular associations, such as hydrophobic interactions, electrostatic interactions, or hydrogen bonds, that are formed during pH shifting that provide protection from swelling and thermal degradation.

Overall, all the NPs showed superior thermal stability at both pH 11 and 7 based upon the small changes in the turbidity, particle size and PDI. This is because the protein was significantly unfolded at pH 11 and the NPs were formed from the aggregation of α-LA and Trp molecules through the surface active groups, with no surface active groups available, during heating, no additional aggregation occurs. The thermal stability of these Trp-containing NPs is a useful property for many commercial applications that involve Trp fortified ingredients in formulations that are exposed to high temperature treatments, especially in beverage and other liquid-type food matrices and nutritional product applications.

TABLE 5
Effect of thermal treatments, freeze-thaw cycle, and freeze-thaw-thermal treatments
on the turbidity and particle size of α-LA-NPs, Trp-NPs, and α-LA-Trp-NPs

| | | pH 11 | | | pH 7 | | |
|---|---|---|---|---|---|---|---|
| | | | Z-average | | | Z-average | |
| Samples ^{a} | Treatments ^{b} | Turbidity | (d · nm) | PDI | Turbidity | (d · nm) | PDI |
| α-LA-NPs | RT | 3.12 ± 0.28 ^{a} | 289.0 ± 3.9 ^{b} | 0.14 ± 0.02 ^{a} | 3.06 ± 0.26 ^{a} | 233.0 ± 12.6 ^{a} | 0.19 ± 0.02 ^{a} |
| | 63° C. 30 min | 3.08 ± 0.27 ^{a} | 445.1 ± 18.7 ^{a} | 0.15 ± 0.04 ^{a} | 3.07 ± 0.26 ^{a} | 241.2 ± 30.2 ^{a} | 0.22 ± 0.01 ^{a} |
| | 90° C. 2 min | 3.11 ± 0.27 ^{a} | 438.4 ± 44.7 ^{a} | 0.15 ± 0.03 ^{a} | 3.16 ± 0.28 ^{a} | 240.1 ± 21.7 ^{a} | 0.21 ± 0.01 ^{a} |
| | Freeze-thaw | 3.18 ± 0.28 ^{a} | 430.1 ± 44.7 ^{a} | 0.23 ± 0.02 ^{a} | 3.07 ± 0.25 ^{a} | 241.7 ± 9.8 ^{a} | 0.19 ± 0.03 ^{a} |
| | Freeze-thaw-heat | 3.06 ± 0.26 ^{a} | 490.8 ± 70.7 ^{a} | 0.22 ± 0.04 ^{a} | 3.08 ± 0.27 ^{a} | 241.7 ± 35.9 ^{a} | 0.23 ± 0.01 ^{a} |
| Trp-NPs | RT | 3.46 ± 0.19 ^{a} | 210.0 ± 9.4 ^{b} | 0.16 ± 0.02 ^{a} | 3.27 ± 0.18 ^{b} | 207.4 ± 4.8 ^{b} | 0.16 ± 0.01 ^{b} |
| | 63° C. 30 min | 3.26 ± 0.18 ^{a} | 212.2 ± 7.7 ^{b} | 0.16 ± 0.03 ^{a} | 3.22 ± 0.19 ^{b} | 204.6 ± 6.6 ^{b} | 0.16 ± 0.02 ^{b} |
| | 90° C. 2 min | 3.31 ± 0.19 ^{a} | 213.0 ± 7.5 ^{ab} | 0.15 ± 0.04 ^{a} | 3.22 ± 0.19 ^{b} | 204.0 ± 3.7 ^{b} | 0.19 ± 0.03 ^{b} |
| | Freeze-thaw | 3.49 ± 0.20 ^{a} | 224.3 ± 4.1 ^{a} | 0.17 ± 0.03 ^{a} | 5.27 ± 0.24 ^{a} | 485.6 ± 253.4 ^{a} | 0.53 ± 0.20 ^{a} |
| | Freeze-thaw-heat | 3.47 ± 0.19 ^{a} | 218.6 ± 3.7 ^{ab} | 0.16 ± 0.02 ^{a} | 5.29 ± 0.08 ^{a} | 335.1 ± 36.7 ^{ab} | 0.44 ± 0.07 ^{a} |
| α-LA-Trp-NPs | RT | 2.87 ± 0.26 ^{a} | 283.0 ± 6.9 ^{c} | 0.16 ± 0.03 ^{a} | 2.69 ± 0.26 ^{a} | 243.0 ± 7.2 ^{a} | 0.18 ± 0.04 ^{a} |
| | 63° C. 30 min | 2.79 ± 0.25 ^{a} | 537.4 ± 55.1 ^{b} | 0.18 ± 0.07 ^{a} | 2.73 ± 0.27 ^{a} | 257.5 ± 22.0 ^{a} | 0.21 ± 0.01 ^{a} |
| | 90° C. 2 min | 2.83 ± 0.25 ^{a} | 515.4 ± 42.9 ^{b} | 0.16 ± 0.04 ^{a} | 2.77 ± 0.27 ^{a} | 253.9 ± 12.9 ^{a} | 0.22 ± 0.06 ^{a} |
| | Freeze-thaw | 2.86 ± 0.25 ^{a} | 324.1 ± 19.1 ^{c} | 0.22 ± 0.02 ^{a} | 2.66 ± 0.22 ^{a} | 248.8 ± 10.3 ^{a} | 0.21 ± 0.04 ^{a} |
| | Freeze-thaw-heat | 2.86 ± 0.23 ^{a} | 501.4 ± 23.1 ^{b} | 0.25 ± 0.02 ^{a} | 2.67 ± 0.23 ^{a} | 249.8 ± 14.5 ^{a} | 0.24 ± 0.05 ^{a} |

Data are means ± standard deviation. Means within columns among the same sample group not sharing a common letter are significantly different at p < 0.05.
^{a} α-LA (α-lactalbumin), Trp (tryptophan), NPs (nanoparticles)
^{b} RT: freshly prepared samples at room temperature
Freeze-thaw: samples undergo a freeze-thaw cycle at −20° C. overnight and then thaw at 25° C.
Freeze-thaw-heat: Samples after freeze-thaw was then subjected to heat treatment at 63° C. for 30 min

### 3.7. Effect of HPH and pH Shifting on the Freeze-Thaw and Freeze-Thaw-Thermal Stability of α-LA-Trp-NPs at Different pH

The physical stability of our NPs-based delivery systems during freeze-thaw and freeze-thaw-thermal cycles is important for products intended for use as food ingredients. We subjected our α-LA-Trp-NPs, α-LA-NPs and Trp-NPs to freeze-thaw and freeze-thaw-thermal stability conditions designed to mimic or exceed food industry conditions. For the freeze-thaw cycle, the samples were subjected to extreme changes in storage temperature from −20° C. to 25° C. and then for the freeze-thaw-thermal cycles the samples underwent an additional heat treatment at 63° C. for 30 min.

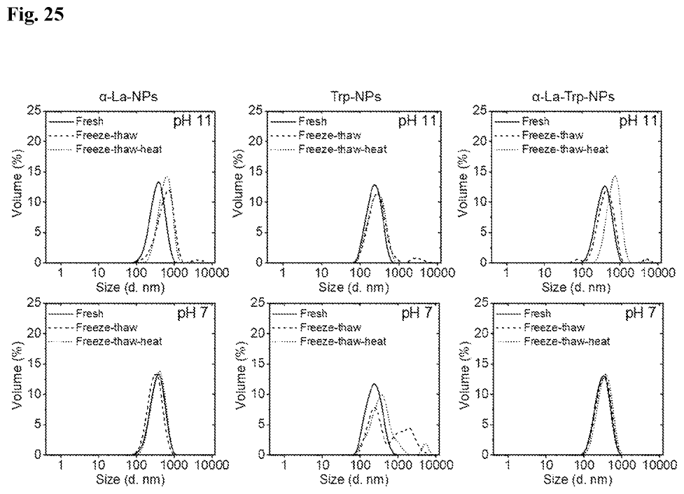

The particle size of the α-LA-NPs increased slightly after the freeze-thaw treatment at pH 11, and then grew again after the heat treatment (FIG. 25 and Table 5). The increase in particle size of proteins after a freeze-thaw has been reported, and it is generally attributed to protein aggregation caused by the growing ice crystals during freezing (Chen et al., (2022). High internal phase Pickering emulsions stabilized by tannic acid-ovalbumin complexes: Interfacial property and stability. Food Hydrocolloids, 125, 107332). The growth of the particle size at pH 11 after heating is consistent with the result for samples without freeze-thaw, where no significant difference was observed in particle size and PDI values (FIG. 24 and Table 5). This may be due to the protein aggregation that occurred during freezing which was subsequently broken down during heating, allowing the NPs to increase in size due to swelling. After pH shifting from pH 11 to pH 7, however, there was no significant change in the particle size of α-LA-NPs after freeze-thaw treatment nor after the freeze-thaw-thermal cycles. This result indicated that the freeze-thaw cycle did not significantly impact the colloidal dispersion stability of α-LA-NPs, and therefore, the particle stability of α-LA-NPs was improved by pH shifting.

The Trp-NPs, conversely, showed markedly different behaviors, exhibiting freeze-thaw stability at pH 11 with poor freeze-thaw stability at pH 7 (FIG. 25). Sedimentation of particles was visually observed after freeze-thaw in Trp-NPs dispersions at pH 7, which is consistent with the increased turbidity (Table 5).

The particle size of α-LA-Trp-NPs at pH 11 was not influenced significantly by the freeze-thaw cycle (FIG. 25 and Table 5). After heat treatment at pH 11, however, the particle size increased slightly, but the size distribution remained narrow. Without wishing to be bound to any particular theory, the presence of Trp in the NPs could increase the freeze-thaw stability of α-LA-Trp-NPs by preventing the aggregation of protein during freezing. Compared to Trp-NPs, there were no visible aggregates observed in «-LA-Trp-NPs dispersion after freeze-thaw and freeze-thaw-thermal treatment at pH 7. At pH 7, the particle size of α-LA-Trp-NPs was uniform and did not change significantly upon freeze-thaw and freeze-thaw-thermal treatment. This suggests that the presence of α-LA increases the freeze-thaw and freeze-thaw-thermal stability of Trp through complex formation via the synergistic effects of HPH and pH shifting.

### 3.8. Re-Dispersibility of Freeze-Dried α-LA-Trp-NPs

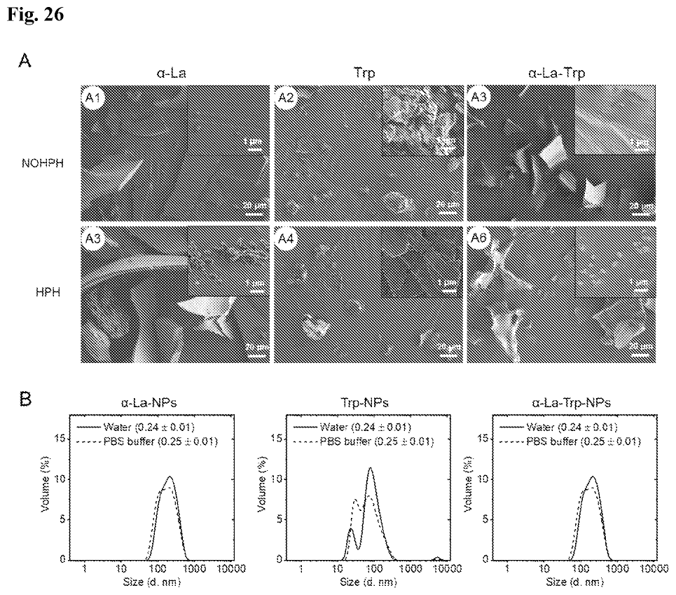

The effect of freeze-drying on our NPs and their re-dispersibility in aqueous solution was investigated. All of the NPs showed signs of aggregation after freeze-drying with only minor differences between the NOHPH and HPH samples (Panel A of FIG. 26). However, small particles were observed on the surface of the aggregates of α-LA (Panel A of FIG. 26) and α-LA-Trp mixture (Panel A of FIG. 26) with HPH. In a similar manner, the self-assembly behavior of tryptophan molecules was observed upon freeze-drying, which resulted in an irregular shape without HPH (Panel A of FIG. 26). However, tryptophan molecules with HPH were observed to be self-assembling into well-ordered networks with nanosheet-like morphology (Panel A of FIG. 26).

We also investigated the redispersion of NPs in aqueous solutions. The dry powder was redispersed in both Milli-Q water and PBS buffer (10 mM, pH 7.0) and their particle size distribution and PDI were determined (Panel B of FIG. 26). All freeze-dried NPs in both Milli-Q water and PBS buffer showed a nanoscale particle size, and the particle size distribution was narrow (PDI<0.3). The mean particle size was slightly smaller for all samples compared with measured size before freeze-drying at pH 7 (Table 4 and FIG. 23), which may be related to the tendency of molecules in NPs to form strong intermolecular associations during freeze-drying and thereby form a strong network within the NPs (Dong et al., 2021). Since the particle size was measured in Milli-Q water and PBS buffer by DLS, the strong network formed during freeze-drying would prevent the hydration of freeze-dried NPs compared to those without freeze-drying and thereby smaller and narrower particle size distribution was observed.

The foregoing is illustrative of the present invention, and is not to be construed as limiting thereof. The invention is defined by the following claims, with equivalents of the claims to be included therein.

That which is claimed is:

1. A particle comprising:
a protein; and
an active agent,
wherein the active agent is present within the protein (e.g., within the tertiary structure of the protein), optionally wherein the active agent is nonspecifically bound (e.g., via hydrophobic interaction, electrostatic interaction, hydrogen bonding, etc.) to the protein.

2. The particle of claim 1, wherein the active agent is within an area of the tertiary structure (e.g., within a tertiary fold) that comprises at least one nonspecific hydrophobic interaction between two or more amino acid residues, optionally wherein the active agent is present in a hydrophobic pocket of the protein.

3. The particle of claim 1 or 2, wherein the active agent is within the protein core of the protein.

4. The particle of any preceding claim, wherein the active agent is a plurality of active agents and at least one active agent of the plurality of active agents is present within the protein, optionally wherein an additional active agent of the plurality of active agents is present on a surface of the protein.

5. The particle of any preceding claim, wherein the protein is selected from a dairy protein (e.g., milk protein), a plant protein, or an animal (e.g., meat) protein, optionally wherein the protein is a milk protein.

6. The particle of any preceding claim, wherein the protein has a molten globule state and/or has a bilobal structure.

7. The particle of any preceding claim, wherein the protein has about 100 amino acids to about 200, 300, 400, or 500 amino acids and/or a molecular weight of about 10,000 kDa to about 20,000, 30,000, 40,000, or 50,000 kDa.

8. The particle of any preceding claim, wherein the protein has an isoelectric point (pI) of about 4 to about 5, optionally wherein the protein has a pI of about 4.2 to about 4.5.

9. The particle of any preceding claim, wherein the protein comprises two domains and/or the protein, at a pH of about 5 to about 9, comprises one or more (e.g., 1, 2, 3, 4, or more) intramolecular disulfide bonds, optionally wherein the protein comprises at least one disulfide bridge that connects two domains of the protein.

10. The particle of any preceding claim, wherein the tertiary structure of protein comprises α-helices in an amount of about 15% to about 30% and α-sheets in an amount of about 5% to about 25%, optionally wherein about 50% to about 75% of the tertiary structure is unordered.

11. The particle of any preceding claim, wherein the protein is selected from α-lactalbumin, lysozyme, cytochrome c, apomyoglobin, and staphylococcal nuclease, optionally wherein the protein is α-lactalbumin.

12. The particle of any preceding claim, wherein the protein has an amino acid sequence having at least 70%, 75%, 80%, 85%, 90%, 95%, 96%, 97%, 98%, 99%, or more sequence identity to one or more of SEQ ID NOs: 1-3.

13. The particle of any preceding claim, wherein the protein is a plurality of proteins, optionally wherein about 5, 10, or 20 to about 25, 30, 40, or 50 proteins are present in the particle.

14. The particle of any preceding claim, wherein the active agent is an organic compound that has a molecular weight of about 70, 100, 150, or 200 g/mol to about 250, 300, 400, or 500 g/mol.

15. The particle of any preceding claim, wherein the active agent has a solubility in water of about 15 mg/mL at 25° C. or less and/or has a pKa of about 1.5 to about 3 and/or a pI of about 5 to about 6.5, optionally wherein the active agent has a solubility in water of about 10 mg/mL at 25° C. or less and/or has pKa of about 2.7 or 2.8 to about 2.9 or 3 and/or a pI of about 5.7 or 5.8 to about 5.9, 6, or 6.1.

16. The particle of any preceding claim, wherein the active agent is selected from tryptophan, leucine, phenylalanine, cysteine, tyrosine, vitamin E, and any combination thereof, optionally wherein the active agent is tryptophan.

17. The particle of any preceding claim, wherein the particle has a diameter of about 25, 50, 100, or 150 nm to about 200, 250, 300, 400, 500, 600, 700, 800, or 900 nm, optionally as measured using microscopy (e.g., scanning electron microscopy (SEM) and/or transmission electron microscopy (TEM)) and/or dynamic light scattering (DLS), optionally wherein the particle has a diameter of about 230 nm.

18. The particle of any preceding claim, wherein the protein is present in the particle in an amount of about 75% to about 100% by weight of the particle and the active agent is present in the particle in an amount of about 1% to about 25% by weight of the particle.

19. The particle of any preceding claim, wherein the protein and the active agent each dissolve in water at a temperature of about 25° C. and a pH of about 11 and/or the protein and/or active agent has a negative charge in water at pH of about 11.

20. The particle of any preceding claim, wherein, upon storage at about 4° C. to about 10° C. in a closed container for about 1, 2, 3, 4, 5, or 6 month(s), the size (e.g., diameter) of the particle remains within ±about 20% of its original size.

21. The particle of any preceding claim, wherein the particle has an increased freeze-thaw stability compared to the freeze-thaw stability of the protein alone.

22. The particle of any preceding claim, wherein, after a change in temperature (e.g., an increase in temperature from about −20, −15, or −10° C. to about 15, 20, or 25° C.), the size (e.g., diameter) of the particle remains within ±about 20% of its original size.

23. The particle of any preceding claim, wherein the particle has an increased freeze-thaw-thermal stability compared to the freeze-thaw-thermal stability of the protein alone.

24. The particle of any preceding claim, wherein, after a change in temperature (e.g., an increase in temperature from about −20, −15, or −10° C. to about 15, 20, or 25° C.) followed by a heat treatment (e.g., a heat treatment at about 55, 60, or 65° C. for about 25, 30, or 35 minutes), the size (e.g., diameter) of the particle remains within ±about 20% of its original size.

25. The particle of any preceding claim, wherein the particle has an increased activity and/or function (e.g., increased antioxidant activity) compared to the activity and/or function of the protein alone.

26. A plurality of particles comprising the particle of any preceding claim.

27. The plurality of particles of claim 26, wherein the plurality of particles have a Dv (50) of about 175 or 200 nm to about 225, 250, 275, 300, or 325 nm.

28. The plurality of particles of claim 26 or 27, wherein the plurality of particles have a polydispersity index (PDI) of less than about 0.5, optionally of less than 0.3 or 0.2.

29. A composition comprising a carrier (e.g., water and/or an oil) and the particle of any one of claims 1-25 or the plurality of particles of any one of claims 26-28, wherein, when the particle or plurality of particles is present in the composition in an amount of about 100 mg per mL of water, less than about 30% of the active agent is present free in the composition.

30. The composition of claim 29, wherein the composition is a dispersion, optionally wherein there is no visible aggregation in the composition (e.g., the composition is clear and is not cloudy or opaque).

31. A method for preparing a particle, the method comprising:
homogenizing or sonicating a composition comprising a protein and an active agent for about 1 minute to about 2 hours, wherein the composition has a pH of about 10 to about 12, thereby providing the particle.

32. The method of claim 31, wherein the method comprises homogenizing the composition at a pressure of about 5,000 psi to about 45,000 psi, optionally wherein the pressure is about 20,000 psi to about 40,000 psi.

33. The method of claim 31, wherein the method comprises sonicating the composition at frequency of about 15 kHz to about 30 kHz with an amplitude of about 40% to about 70%.

34. The method of any one of claims 31-33, wherein the composition comprises the protein and active agent in a weight ratio of about 2:1 to about 30:1 (protein:active agent), optionally wherein the weight ratio is about 5:1 to about 20:1 (protein:active agent).

35. The method of any one of claims 31-34, wherein the composition has a solids content of about 1% w/v to about 20% w/v.

36. The method of any one of claims 31-35, wherein the homogenizing or sonicating is carried out at a temperature in a range from about 15° C. to about 30° C. for about 10 minutes to about 50 minutes.

37. The method of any one of claims 31-36, further comprising, after homogenizing or sonicating the composition, adjusting the pH of the composition to about 6.5 to about 7.5, optionally to a pH of about 7.

38. The method of any one of claims 31-37, wherein the particle has a particle size distribution in a range of about 50, 100, 200, or 300 nm to about 400, 500, 600, or 700 nm with a polydispersity index of less than about 0.5, 0.45, 0.4, 0.35, 0.3, 0.25, or 0.2 and/or wherein the particle has a mean particle size (e.g., mean particle diameter) of about 100, 125, 150, or 175 nm to about 200, 225, 250, 275, or 300 nm.

39. The method of any one of claims 31-38, further comprising dehydrating the particle, optionally wherein dehydrating the particle comprises freeze-drying or spray-drying the composition.

40. The method of any one of claims 31-39, further comprising reducing the size (e.g., diameter) of the particle, optionally wherein the size of the particle is reduced by about 5% to about 40% compared to the size of the particle in the composition at pH of about 11.

41. The method of any one of claims 31-40, wherein the particle is a particle of any one of claims 1-25 and/or wherein the method provides the plurality of particles of any one of claims 26-28 and/or the composition of claim 29 or 30.

42. An article comprising the particle of any one of claims 1-25, the plurality of particles of any one of claims 26-28, the composition of claim 29 or 30, and/or the particle prepared according to any one of claims 31-41.

43. The article of claim 42, wherein the article is a food product (e.g., infant formula, a dairy product, etc.), nutritional supplement, therapeutic drink, and/or cosmetic.


SEQUENCE LISTING (3 sequences)

SEQ ID NO: 1 - protein, 123 aa, Bos taurus
EQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 2 - protein, 162 aa, Bos taurus
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 3 - protein, 708 aa, Bos taurus
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR